(19) United States
(12) Patent Application Publication
(10) Pub. No.: US 2025/0057979 A1
(43) Pub. Date: Feb. 20, 2025

(54) CARDIAC CALCIUM HANDLING MACHINERY REMODELED IN FRIEDREICH'S ATAXIA

(71) Applicant: University of South Florida, Tampa, FL (US)

(72) Inventor: Bojjibabu Chidipi, Tampa, FL (US)

(21) Appl. No.: 18/668,193

(22) Filed: May 19, 2024

Related U.S. Application Data
(60) Provisional application No. 63/519,318, filed on Aug. 14, 2023.
(60) Provisional application No. 63/595,975, filed on Nov. 3, 2023.

(51) Int. Cl. A61K 48/00; A61K 31/055; A61K 31/47; A61K 31/566; A61K 38/17; A61K 45/06; A61P 9/06; A61P 25/28; C12N 15/86; G01N 33/543; G01N 33/68

(52) U.S. Cl. CPC A61K 48/005; A61K 31/055; A61K 31/47; A61K 31/566; A61K 38/177; A61K 45/06; A61P 9/06; A61P 25/28; C12N 15/86; G01N 33/543; G01N 33/6893; C12N 2750/14143; G01N 2333/705

(57) ABSTRACT

The present disclosure provides for a method of treating Friedrich's Ataxia in a subject in need thereof comprising administering to the subject a composition to upregulate type 2 ryanodine receptors (Ryr2), a composition to activate Ca²⁺ release, a composition to upregulate SERCA2a, a composition to upregulate Phasoplamban (PLN), a composition to upregulate Ca²⁺/calmodulin-dependent protein kinase II (CaMKII), or any combination thereof. Further provided herein is a method of diagnosing Friedrich's Ataxia and a kit related thereto.

### CROSS-REFERENCE TO RELATED APPLICATIONS

This application claims the benefit of priority to, and the benefit of, U.S. Provisional

Application No. 63/519,318, filed on Aug. 14, 2023, and U.S. Provisional Application No. 63/595,975, filed on Nov. 3, 2023, the disclosures of which are hereby expressly incorporated by reference herein in their entireties.

### BACKGROUND

Friedreich's ataxia (FA) is an inherited neurodegenerative disorder that causes progressive nervous system damage resulting in impaired muscle coordination. FA affects almost 1 in 50,000 people with equal frequency in both sexes and is typically diagnosed in mid-childhood. FA is the most common autosomal recessive form of ataxia and is caused by an expansion of the DNA triplet guanine-adenine-adenine (GAA) in the first intron of the Frataxin gene (FXN), located on chromosome 9q13. In the unaffected population, the number of GAA repeats ranges from 6 to 27 repetitions. In FA patients, GAA repeat expansions range from 44 to 1,700 repeats, which decreases frataxin protein expression. Frataxin is a 210 amino acid mitochondrial protein essential for various cellular functions, including iron metabolism. Reduced frataxin expression is thought to negatively affect mitochondrial iron metabolism, leading to increased oxidative damage. Although FA is considered a neurodegenerative disorder, FA patients display heart disease that includes hypertrophy, heart failure, arrhythmias, conduction abnormalities, and cardiac fibrosis. It is thought that decreased expression of frataxin, as in FA, impairs mitochondrial function by impairing the iron-sulfur cluster synthesis, therefore affecting heme biosynthesis, and ultimately leading to increased oxidative stress. Decreased limbs and trunk movements, dysarthria, and heart disorders are clinical symptoms of FA.

The compositions and methods disclosed herein address these and other issues resulting from FA.

### SUMMARY

In accordance with the purposes of the disclosed materials and methods, as embodied and broadly described herein, the disclosed subject matter, in one aspect, relates to Friedrich's Ataxia and methods of treating thereof.

Thus, in one example, a method of treating Friedrich's Ataxia in a subject in need thereof is provided, including administering to the subject a composition to upregulate type 2 ryanodine receptors (Ryr2), a composition to activate Ca²⁺ release, a composition to upregulate SERCA2a, a composition to upregulate Phasoplamban (PLN), a composition to upregulate Ca²⁺/calmodulin-dependent protein kinase II (CaMKII), or any combination thereof.

In a further example, a method of diagnosing Friedrich's Ataxia in a subject is provided, including assaying Ryr2, SERCA2a, PLN, CaMKII, or any combination thereof; and administering the composition disclosed herein to the subject wherein the subject has decreased Ryr2, PLN, CaMKII, and/or SERCA2a levels as compared to an unaffected control.

Additionally, a kit is provided, including (1) a device for screening a sample from a subject for Ryr2 and (2) a composition to upregulate Ryr2, a composition to activate Ca²⁺ release, a composition to upregulate SERCA2a, a composition to upregulate Phasoplamban (PLN), a composition to upregulate Ca²⁺/calmodulin-dependent protein kinase II (CaMKII) or any combination thereof.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIGS. 1A-1B show QT prolongation in 9.5 weeks old FXN-KO mice. (FIG. 1A) Representative ECG tracings for FXN-WT and KO mice inserts are single representative traces. (FIG. 1B) Summary data comparing the duration of RR, PR, QRS, QT, and QTc intervals from WT vs. KO mice. QT and QTc were significantly increased in FXN-KO compared to the FXN-WT **p<0.01 for FXN-WT vs. FXN-KO. Bars are mean±SEM.

FIGS. 2A-2B show abnormal ECG in 12 weeks old FXN-KO mice. (FIG. 2A) Representative ECG tracings for FXN-WT and KO mice, inserts are single representative traces. (FIG. 2B) Summary data comparing the duration of (a) RR, (b) PR, (c) QRS, (d) QT and QTc intervals from WT vs. KO micc. **p<0.01 for WT vs. KO. Bars are mean±SEM.

FIGS. 3A-3B show echocardiography measurements of FXN-WT and FXN-KO mouse hearts. (FIG. 3A) B and M-mode images. (FIG. 3B) Summary bar graphs of (a) Left ventricular ejection fraction (EF), (b) Fractional shortening (FS), (c) Left ventricular end-systolic volume, (d) Left ventricular end-diastolic volume. (*p<0.05, **p<0.01, vs. FXN-WT; unpaired 2-sample t-test). Bars are mean±SEM.

FIGS. 4A-4D show Western blot analysis of major Ca 2+ handling proteins levels in FA. Representative blots of sarco-endoplasmic reticulum ATPase 2a (SERCA2a), and ryanodine receptor (RyR2) in FXN-WT and KO (FIG. 4A), and control and FA (FIG. 4C) ventricular tissue samples. Barographs are quantification of SERCA2a and RyR2 normalized by GAPDH. SERCA2a and RyR2 were significantly decreased in FXN-KO compared to the WT (FIG. 4B) and in FA compared to the control (FIG. 4D). (**p<0.01 vs. control or WT; unpaired 2-sample t-test). Bars are mean±SEM.

FIGS. 5A-5D show sarcomere and cell shortening in isolated ventricular cardiomyocytes from FXN-WT and KO mice. (FIG. 5A) Representative traces of sarcomere shortening, (FIG. 5B) Summary of sarcomere shortening was significantly less in FXN-KO and WT, (FIG. 5C) Representative traces of cell shortening, and (FIG. 5D) Summary of cell shortening was significantly less in FXN-KO and WT. (*p<0.05, vs WT; unpaired 2-sample t-test). Bars are mean±SEM.

FIGS. 6A-6H show mitochondrial perturbations in FA (FIG. 6A) Visualization of TMRM ΔΨm fluorescence in FXN-WT and KO cardiomyocytes. (FIG. 6B) Time-dependent ΔΨm over 22 min, after 15 min, the addition of 10 μM of FCCP decreased ΔΨm. (FIG. 6C) Average ΔΨm intensity was significantly less in FXN-KO (n=6). (FIG. 6D) Representative bright field and MitoSOX fluorescence images of cardiomyocytes from FXN-WT and FXN-KO. (FIG. 6E) Relative MitoSOX fluorescence was significantly higher in FXN-KO compared to the FXN-WT (n=9). (FIG. 6F) Oxygen consumption rate (OCR) in FXN-WT and FXN-KO, the curve shows the change in OCR after injections of Oligo (oligomycin), FCCP (phenylhydrazone), and A/R (Antimycin and Rotenone). (FIG. 6G) Quantification of normalized Max (maximal) OCR, ATP (ATP linked) OCR, and nMTO (non-mitochondrial) OCR (n=9). (FIG. 6H) Electron micrographs of cardiac muscle from FXN-WT and KO ventricle tissues. Left-side images were captured with 10,000× magnification, and right-side images were selected area of left-side images captured with 100,000× magnification shows normal mitochondria with healthy cristac FXN-WT and over-fused mitochondrial with disturbed cristac in FXN-KO. (**p<0.01, vs. WT; unpaired 2-sample t-test). Bars are mean±SEM.

FIGS. 7A-7B show proteomic analysis in FA mouse and human hearts. FIG. 7A shows significantly upregulated and downregulated proteins in human hearts with FA (N=5) compared to controls (N=5) and in FA mouse hearts (N=3) versus controls (N=3). FIG. 7B is an illustration of the “cardiac muscle contraction” KEGG pathway with the differentially regulated proteins in human (dark grey stars) and mouse (lighter gray stars) FA versus control left ventricular lysates.

FIGS. 8A-8D show Western blot analysis of major Ca 2+ handling proteins levels in FA. Representative blots of sarco-endoplasmic reticulum ATPase 2a (SERCA2a) and ryanodine receptor (RyR2) in FXN-WT and KO (FIG. 8A), and human control and FA (FIG. 8C) ventricular tissue samples. Barographs are quantification of SERCA2a and RyR2 normalized to GAPDH. SERCA2a and RyR2 were significantly decreased in FXN-KO compared to the WT (FIG. 8B) and in human FA samples compared to the control (FIG. 8D). (**p<0.01 vs. control or FXN-WT; unpaired 2-sample t-test). Bars are mean±SEM.

FIGS. 9A-9D show Phasoplamban (PLN) and p-PLN proteins expression in FA human and mouse ventricular tissue. FIG. 9A and FIG. 9B are the representative western blots of PLN and p-PLN expression in FA patients and control ventricular tissues. FIG. 9C and FIG. 9D are the densitometric analysis of PLN and p-PLN at Ser16 and Th17 normalized to GAPDH. The monomeric and pentameric forms of PLN protein expression significantly decreased in FXN-KO lysates compared to the WT, and the decrease of PLN protein expression was not significant in FA hearts compared to the control (PLN-monomeric p=0.054 and PLN-pentameric p=0.59). The p-PLN of monomeric at the Ser16 and Thr17 sites were significantly decreased in FA and FXN-KO hearts compared to the control and WT. (*p<0.05 and **p<0.01; unpaired 2-sample t-test).

FIG. 10 shows mitochondrial Ca²⁺ gating proteins expression in FA-KO vs WT. The upper panels are representative blots of MCU, VDAC, NCLX, and GAPDH. Bar graphs show that MCU and VDAC were significantly changed in FA mice, compared to the WT (*p<0.05; unpaired 2-sample t-test).

FIGS. 11A-11D show Sarcomere and cell shortening in isolated ventricular cardiomyocytes from FXN-WT and KO mice. (FIG. 11A) Representative traces of sarcomere shortening, (FIG. 11B) Summary of sarcomere shortening in FXN-KO and WT, (FIG. 11C) Representative traces of cell shortening, and (FIG. 11D) Summary of cell shortening in FXN-KO and WT. (*p<0.05, vs FXN-WT; unpaired 2-sample t-test). Bars are mean±SEM.

FIG. 12 shows abnormal ECG in FXN-KO mice with summary data comparing the duration of RR, PR, QRS, QT, and QTc intervals from WT vs. KO mice.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed. When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

The term “subject” preferably refers to a human in need of treatment with an anti-cancer agent or treatment for any purpose, and more preferably a human in need of such a treatment to treat cancer, or a precancerous condition or lesion. However, the term “subject” can also refer to non-human animals, preferably mammals such as dogs, cats, horses, cows, pigs, sheep and non-human primates, among others, which are in need of treatment with an anti-cancer agent or treatment.

The term “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Method

### Method of Treating Friedrich's Ataxia (FA)

The present disclosure, in one aspect, provides for a method of treating Friedrich's Ataxia in a subject in need thereof comprising administering to the subject a composition to upregulate type 2 ryanodine receptors (Ryr2), a composition to activate Ca²⁺ release, a composition to upregulate SERCA2a, a composition to upregulate Phasoplamban (PLN), a composition to upregulate Ca²⁺/calmodulin-dependent protein kinase II (CaMKII), or any combination thereof.

Friedrich's Ataxia (FA) is an inherited disorder that impacts some of the body's nerves. It is caused by a gene defect inherited from both parents. In some examples, symptoms begin in late childhood and can include trouble walking, fatigue, changes in sensation, and slowed speech. In further examples, these symptoms tend to get worse over time. In specific examples, heart disease, skeletal problems, diabetes, or any combination thereof can also occur. There is currently no cure for FA but treatments to treat the disease and symptoms thereof include surgery and physical, occupational, and speech therapy. Ryanodine receptor 2 (Ryr2) is one of a class of ryanodine receptors and a protein found at least in cardiac muscle. In humans, it is encoded by the RYR2 gene. In the process of cardiac calcium-induced calcium release, Ryr2 is a major mediator for sarcoplasmic release of stored calcium ions. The Ryr2 protein functions as a notable component of a calcium channel located in the sarcoplasmic reticulum that supplies ions to the cardiac muscle during systole.

Calcium-induced calcium is used by most cells to amplify Ca²⁺ signals. In heart cells, this mechanism operates between voltage-gate Ca²⁺ channels, which are located in the plasma membrane and ryanodine receptor channels, which are located in the sarcoplasmic reticulum. The calcium-induced calcium release produced much needed calcium for the contraction of the heart.

SERCA2a refers to the sarcoplasmic/endoplasmic reticulum Ca²⁺ ATPase 2a which is a subtype of SERCA expressed in the heart. SERCA2a mediates the contraction of cardiomyocytes and Ca²⁺ in the cytoplasm to re-enter in the sarcoplasmic reticulum. SWRCA2a is encoded by the ATP2A2 gene.

Phospholamban (PLN) is a micropeptide protein that is encoded in humans by the PLN gene. Phospholamban is a 52-amino acid integral membrane protein and regulates the calcium pump in cardiac muscle cells. PLN is found as a pentamer and is a major substrate for the cAMP-dependent protein kinase in cardiac muscle. In its unphosphorylated state, PLN is an inhibitor of cardiac muscle sarcoplasmic reticulum Ca²⁺-ATPase (SERCA2). Ca²⁺/calmodulin-dependent protein kinase II (CaMKII) is a serine/threonine-specific protein kinase that is regulated by the Ca²⁺/calmodulin complex. It is involved in signaling cascades and utilized for Ca²⁺ homeostasis and reuptake in cardiomyocytes. Isoforms of CaMKII have a catalytic domain, an autoinhibitory domain, a variable segment, and a self-association domain. The sensitivity of the CaMKII enzyme to calcium dn calmodulin is governed by the variable and self-associative domains.

In some examples, the subject has a cardiac disease. Cardiac disease is a general term that refers to disease of the heart or blood vessels.

In further examples, the cardiac disease is hypertrophic cardiomyopathy, atrial tachycardia, intra atrial block, or any combination thereof.

Hypertrophic cardiomyopathy is a condition affecting the left ventricle, the main pumping chamber of the heart. The walls of the left ventricle become thick and stiff and over time, the heart is not able take in or pump out sufficient blood during each heartbeat to supply the body's needs.

Atrial tachycardia (AT) is a type of abnormal heart rhythm, or arrythmia, AT occurs when the electrical signal that controls the heartbeat starts from an abnormal location in the atria and rapidly repeats, causing the atria to beat too quickly. It is a type of supraventricular tachycardia (SVT). During an AT episode, the heart beats more than 100 times a minute and then returns to a heart rate of around 60 to 80 beats a minute. In some examples, an episode starts slowly, while in other examples, an episode may start suddenly or quickly. It can cause a pounding or racing heartbeat, light-headedness, dizziness, and/or fainting.

Intra atrial block (IAB) is characterized by the shape of the P-wave in an ECG. In one type of IAB, the P-wave is widened, with or without notching. In another type of IAB, the P-wave has abrupt and transient P-wave widening. In a further type of IAB, there is loss of conduction.

In certain examples, the subject exhibits reduced left ventricular ejection fraction. Left ventricular ejection fraction (LVEF) refers to the central measure of left ventricular systolic function. It is the fraction of chamber volume ejected in systole (stroke volume) in relation to the volume of blood in the ventricle at the end of diastole (end-diastolic volume). Stroke volume is calculated as the difference between end-diastolic volume and end systolic volume. LVEF levels are categorized into five categories: hyperdynamic, normal, mild dysfunction, moderate dysfunction, and severe dysfunction. Hyperdynamic refers to a LVEF greater than 70%. Normal refers to an LVED of 50% to 70%. Mile dysfunction refers to an LVEF of 40% to 49%. Moderate dysfunction refers to an LVEF of 30% to 39%. Severe dysfunction refers to an LVEF of less than 30%.

In some examples, a reduced LVEF refers to an LVEF of 49% or less.

In specific examples, the composition to activate Ca²⁺ release comprises 4-chloro-m-cresol. 4-chloro-m-cresol, also referred to as 4-chloro-3-methylphenol, is a ryanodine receptor agonist. It is a hydroxytoluene and a member of monochlorobenzenes. It has the following chemical structure:

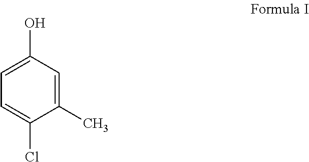

In some examples, the composition to upregulate SERCA2a comprises isatroxime,

CDN1163, or any combination thereof.

Isatroxime is a pharmaceutical drug that acts as a positive inotropic agent, mediating its action through inhibition of sodium/potassium adenosine triphosphatase. Isatroxime is also referred to as (3Z,5a)-3-[(2-Aminoethoxy) imino] androstane-6,17-dione. Isatroxime has the following chemical structure:

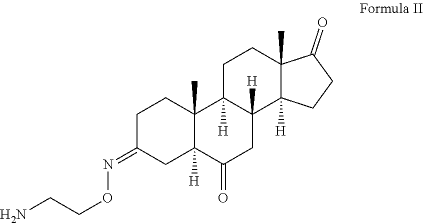

CDN1163 is an allosteric sarco/endoplasmic reticulum Ca²⁺-ATPase (SERCA) activator that improves Ca²⁺ homeostasis. CDN1163 has the following chemical structure:

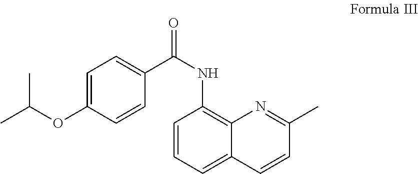 [SMILES: Cc1ccc2cccc(NC(=O)c3ccc(OC(C)C)cc3)c2n1]

In further examples, the composition to upregulate SERCA2a comprises a pyridine derivative.

Pyridine is an azaarene comprising a benzene core in which one —CH group is replaced by a nitrogen atom. It has the following chemical structure:

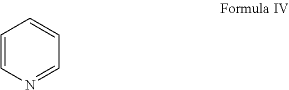

Pyridine derivatives are drugs comprising the pyridine compound and include, but are not limited to, amlodipine, nifedipine, levamlodipine, isradipine, nimodipine, nisoldipine, lercanidipine, nicardipine, felodipine, nitrendipine, nilvadipine, barnidipine, benidipine, cilnidipine, darodipine, lacidipine, manidipine, niguldipine, and niludipine. In certain examples, the composition to upregulate SERCA2a comprises AAVI/SERCA2a gene therapy. AAVI/SERCA2a gene therapy refers to the gene transfer of SERCA2a driven by AAV1-CMV-SERCA2a, thereby upregulating SERCA2a in a patient.

In specific examples, the composition to upregulate Ryr2 comprises mRNA gene therapy to overexpress Ryr2. Ryr2 is decrease in FA related heart failure and therefore, in some examples, administration of mRNA gene therapy with an overexpression of Ryr2upregulates Ryr2 in the subject.

### Method of Diagnosing Friedrich's Ataxia

Further provided herein is a method of diagnosing Friedrich's Ataxia in a subject comprising assaying Ryr2, SERCA2a, PLN, CaMKII, or any combination thereof; and administering the composition of claim 1 to the subject wherein the subject has decreased Ryr2, PLN, CaMKII, and/or SERCA2a levels as compared to an unaffected control.

In some examples, assaying for Ryr2 comprises performing a Western blot test. The Western blot test is an analytic technique used to detect specific proteins in a sample of tissue homogenate or extract. This technique can also be used to visualize, distinguish, and quantify different proteins in a protein combination. Western blot used the following three elements to separate a specific protein from a sample: separation by size, transfer of protein to a solid support, and marking of a target protein using an antibody to visualize. A synthetic or animal-derived antibody is created that recognizes and binds to a specific target protein (the primary antibody). A secondary antibody is added which recognizes and binds to the primary antibody. Using varying methods such as staining, immunofluorescence, and radioactivity, for example, the user can indirectly detect the specific target protein.

In further examples, performing the Western blot test comprises contacting the sample with an antibody, wherein the antibody is IgG kappa BP-HRP. The IgG kappa BP-HRP antibody is a kappa binding protein conjugated to Horseradish Peroxidase. In some examples, this antibody is derived from a mouse. In further examples, the antibody is used in immunochemistry. In certain examples, this antibody is used for Western blot analysis.

In certain examples, the sample comprises a cell, a tissue, urine, blood, semen, plasma, feces, bone marrow, saliva, or any combination thereof from the subject.

In specific examples, assaying the sample from the subject for Ryr2, PLN, CaMKII, and/or SERCA2a, or any combination thereof comprises analyzing the sample via mass spectrometry, performing an enzyme-linked immunosorbent assay (ELISA), performing a protein array, or performing a reverse-phase protein array.

Mass spectrometry (MS) analysis of proteins measure the mass-to-charge ratio of ions to identify and quantify molecules in mixtures and samples of varying complexities. MS can be used to determine protein structure, function, folding, and interactions, identify a protein from the mass of its peptide fragments, and monitor enzyme reactions, chemical modifications, and protein digestion, for example.

ELISA is a plate-based assay technique for detecting and quantifying soluble substances, such as peptides, proteins, antibodies, and hormones. In an ELISA< an antigen (target macromolecule) is immobilized on a solid surface (e.g., microplate) and then complexed with an antibody that is linked to a reporter enzyme. Detection is accomplished by measuring the activity of the reporter enzyme via incubation with the appropriate substrate to produce a measurable product. ELISA utilizes a specific antibody-antigen interaction.

A protein array, or protein microarray, is a high-throughput method of tracking interaction and activities of proteins, as well as determining their function. The protein array includes a substrate surface, such as a glass slide, nitrocellulose membrane, bead, or microtiter plate, for example, to which an array of capture proteins is bound. Probe molecules, in some examples a fluorescent dye, are added to the array and any resulting reaction between the probe and immobilized protein emits a fluorescent signal read by a laser scanner.

Reverse-phase protein array (RPPA) is a high-throughput antibody-based targeted proteomics platform that has the ability to quantify hundreds of proteins in thousands of samples derived from tissue or cell lysates, serum, plasma, or other body fluids. Protein samples are arrayed as microspots on nitrocellulose-coated glass slides and each slide is probed with a specific antibody that can detect levels of total protein expression or post-translational modification, such as phosphorylation as a measure of protein activity.

### Kit

Provided herein is a kit comprising (1) a device for screening a sample from a subject for Ryr2 and (2) a composition to upregulate Ryr2, a composition to activate Ca²⁺ release, a composition to upregulate SERCA2a, a composition to upregulate Phasoplamban (PLN), a composition to upregulate Ca²⁺/calmodulin-dependent protein kinase II (CaMKII) or any combination thereof. In some examples, the composition comprises 4-chloro-m-cresol, isatroxime, CDN1163, a pyridine derivative, AVVI/SERCA2a gene therapy, mRNA gene therapy to overexpress Ryr2, or any combination thereof. In further examples, the device for screening a sample comprises a Western blot test. In certain examples, the subject has Friedrich's Ataxia.

In specific examples, the kit further comprises an antibody for the Western blot test, wherein the antibody is IgG kappa BP-HRP.

In some examples, the sample comprises a cell or tissue from the subject.

In further examples, the device for screening a sample comprises any of the assaying devices disclosed herein.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

### The Cardiac Calcium (Ca²⁺) Handling Machinery is Remodeled in Friedreich's Ataxia

### Introduction

Investigated herein was whether abnormal Ca²⁺ handling machinery is the molecular mechanism that perpetuates cardiac dysfunction in FA.

Used herein was the frataxin knock-out (FXN-KO) mouse model of FA as well as human heart samples from donors with FA and from unaffected donors. ECG and echocardiography were used to assess cardiac function in the mice. Expression of calcium handling machinery proteins was assessed with proteomics and western blot. In left ventricular myocytes from FXN-KO and FXN-WT mice, the lonOptix system was used for calcium imaging, the seahorse assay was utilized to measure oxygen consumption rate (OCR), and confocal imaging was used to quantify the mitochondrial membrane potential (ΔΨm) and reactive oxygen species (ROS).

It was found herein that the major contractile proteins, including SERCA2a and Ryr2, were downregulated in Western blotting analysis of human left ventricular samples from deceased donors with FA compared to unaffected donors, similar to the downregulation of these proteins in the left ventricular tissue from FXN-KO compared to FXN-WT. On the ECG, the RR, PR, QRS, and QTc were significantly longer in the FXN-KO mice compared to FXN-WT. The ejection fraction and fractional shortening were significantly decreased and left ventricular wall thickness and diameter were significantly increased in the FXN-KO mice versus FXN-WT. The mitochondrial membrane potential ΔΨm was depolarized, ROS levels were elevated, and OCR was decreased in ventricular myocytes from FXN-KO versus FXN-WT.

The development of left ventricular contractile dysfunction in FA is feasibly associated with reduced expression of calcium handling proteins and mitochondrial dysfunction.

In FA, the leading causes of mortality are cardiac dysfunction (59%), non-cardiac abnormalities (27.9%), and unknown (9.8%) causes. Hypertrophic cardiomyopathy (HCM) is the most commonly occurring cardiac disease in FA. Left ventricle (LV) wall thickness, LV mass, and LV ejection fraction are significantly changed in FA HCM. Additionally, atrial tachycardia, and intra atrial block are also found in FA patients. The interatrial block most likely leads to the increased P-wave duration that is manifested in FA. Norrish G et al. conducted a study on a large cohort of FA patients and found that heart failure is a major cause of death in this patient population. Additionally, the development of left ventricular systolic dysfunction has been shown to lead to a poorer prognosis in FA patients. Despite the strong cardiac phenotype associated with FA, there has previously been no solid understanding of the progression of cardiac abnormalities in FA.

Ca²⁺ plays a notable role in the regulation of cellular function as a major intracellular messenger. Intracellular changes in Ca²⁺ concentration regulate cardiac contractility. During systole, Ca²⁺ ions enter the myocyte through L-type Ca²⁺ channels located mainly in the transverse tubules. This leads to the release of Ca²⁺ from the sarcoplasmic reticulum (SR) through the type 2 ryanodine receptors (Ryr2) in a process known as Ca²⁺ induced Ca²⁺ release. The free Ca²⁺ then binds to troponin C which allows for the cross-bridge formation of contractile machinery and leads to muscle contraction. This free Ca²⁺ can also be taken into the mitochondria for the generation of ATP via activation of cellular processes, such as the citric acid cycle and oxidative phosphorylation. Ca²⁺ transport to and from the mitochondria is tightly regulated by several membrane proteins, including voltage-dependent anion channel (VDAC), mitochondrial Ca²⁺ uniport (MCU), and the mitochondrial sodium-Ca²⁺ exchanger (NCLX).

During diastole, Ca²⁺ is then removed from the cytoplasm for relaxation to occur, where Ca²⁺ is pumped back into the SR by the SERCA2a and extruded out of the cell through the sodium-Ca²⁺ exchanger (NCX). Alterations or mutations in Ca²⁺ handling proteins, such as Ryr2, SERCA2a, and NCX can result in cardiac pathology and arrhythmias. Additionally, irregularities in mitochondrial machinery can be detrimental to cardiomyocyte survival, resulting in progression to heart failure.

The correlation between cardiac involvement in FA and the decrease of frataxin affecting cardiac contractility has not previously been analyzed. It was investigated herein whether the Ca²⁺ handling machinery is remodeled in FA, and it was found that Ca²⁺ handling was dysregulated, and cardiac contractility and cellular function were decreased. Methods

### FA Animal Model

Used herein was the FA mouse model B6.Cg-Fxn^{em2Lutzy} Fxn^{em2.1Lutzy}Tg (Ckmm-cre)5Khn/J (FXN-KO) and littermates control Heterozygous for Fxn^{em2Lutzy}, Wildtype for Fxn^{em2.1Lutzy}, Hemizygous for Tg(Ckmm-cre) 5Khn (FXN-WT) purchased from the Jackson Laboratory (Stock #029720). Generated herein were these FXN-KO and FXN-WT mice (Stock #029720) by breeding male C57BL/6J-congenic Fxn^{em2Lutzy} (homozygous frataxin floxed exon 2) (Stock #028520) and female C57BL/6-congenic Fxn^{em2.1Lutzy} Tg (Ckmm-cre) 5Khn (heterozygous frataxin global knockout and homozygous MCK-Cre) (Stock #029100), and these breeders were purchased from the Jackson Laboratory. For euthanasia, CO2 was used to induce loss of consciousness, and after the loss of the pinch reflex, swift cervical dislocation was performed, followed by rapid harvesting of the heart via thoracotomy. This method is consistent with the guidelines of the American Veterinary Medical Association.

### Human Ventricular Tissues

Left ventricular samples were obtained from five different decedent donors with FA from Friedreich's Ataxia Research Alliance (FARA) tissue bank at Albany VA Hospital. Left ventricular samples were also obtained from five age, and sex matched samples from donors without an FA diagnosis. These control hearts were rejected for transplant and were obtained through a USF agreement with Lifelink, an FDA-approved, Tampa based organization for organ procurement (listed in Table 1). ECG Mice were anesthetized with 1.8% isoflurane. The ECG was recorded in Lead II configuration via an Animal Bio Amp (AD Instruments, USA) and digitized with the PowerLab data acquisition system (AD Instruments, USA). LabChart Pro 7.2 software (AD Instruments, USA) was used for the acquisition and analysis of cardiac electrical signals.

TABLE 1
Patient heart identification, gender, and age.

| | | |
|---|---|---|
| Name | Gender | Age |

Control Hearts

| | | |
|---|---|---|
| 1 | M | 68 |
| 2 | F | 68 |
| 3 | F | 29 |
| 4 | M | 36 |
| 5 | F | 30 |

FA Patient's Hearts

| | | |
|---|---|---|
| 1 | M | 80 |
| 2 | F | 67 |
| 3 | F | 26 |
| 4 | M | 35 |
| 5 | F | 28 |

### Echocardiography

Transthoracic echocardiography was performed using a Vevo3100LT Ultrasonograph (Visual Sonics) equipped with a 30 MHz transducer. Cardiac function was measured in both FXN-WT and FXN-KO mice. Briefly, the mice were anesthetized with 1-1.5% isoflurane, and body temperature was maintained at 37° C. First, a 2-D mode in the parasternal long axis was imaged to identify the left ventricle part and then switched to the parasternal short axis at the mid-papillary muscle to image the thickness of the wall. From this parasternal short-axis view, the 2-D guided M-mode across the anterior wall and posterior wall were recorded. Left ventricular anterior wall (LVAW), left ventricular interior diameter (LVID), and left ventricular posterior wall thickness (LVPW) at systole and diastole were measured. Ejection fraction (EF) % was calculated as (EDV-ESV)/EDV*100%, and fractional shortening (FS) % was calculated by (LVID;d-LVID;s)/LVID;d*100%.

Western Blotting

For collecting lysates from the left ventricle, samples from FXN-KO, FXN-WT, FA human, and control ventricular tissues were flash frozen in liquid nitrogen, and tissues were cryo-powdered with a Bessman tissue pulverizer (Spectrum Med, San Diego, CA, USA). Protein isolation was completed by adding 10 μL/mg of a RIPA lysis buffer to the cryopowder. The total lysate was centrifuged at 12,000 rpm for 20 min, and the supernatant was collected. Protein concentrations were determined with a BCA assay (Pierce™ BCA Protein Assay Kit; #23225, ThermoFisher, Carlsbad, CA, USA). Total protein (5˜15 μg) was loaded and separated by 4-20% premade SDS-PAGE gels (#4568095, BioRad, Mini-PROTEAN TGX Stain-Free Precast Gels, Irvine, CA, USA), then transferred to a nitrocellulose membrane, and probed with an antibody against the following primary antibodies: mouse monoclonal anti-Frataxin (dilution 5 μg/ml; Abcam; Catalog #ab110328), and mouse monoclonal anti-Ryr2 (dilution 1:1000; Santa Cruz Biotechnology; Catalog #sc-376507), mouse monoclonal anti-SERCA2a (dilution 1:500; Santa Cruz Biotechnology; Catalog #sc-376235). The following secondary antibodies were used; mouse IgG kappa BP-HRP (dilution 1:500; Santa Cruz Biotechnology; Catalog #sc-516102) for detecting Ryr2, fluorescent IRDye 680 goat anti-mouse antibodies (1:25,000; LiCOR; Catalog #925-68072) for SERCA2a for 1 hr. at room temperature. The final images were detected with LiCOR Odyssey, and chemo doc and protein bands were quantified using Imagestudio-LiCOR, software-2019, and FIJI-ImageJ.

### Single-Cell Isolation

Ventricular myocytes were enzymatically dissociated from young and old WT and KO mice. In short, immediately after cardiac excision, the heart was cleaned, and the aorta was cannulated. The heart was then retrogradely perfused at 2 mL/min at 36±0.5° C., for 3 min with Ca²⁺ free Tyrode solution (in mM): NaCl 137, KCl 5.4, HEPES 10, MgCl2 1, and glucose 10 (pH 7.3) until the effluent was clear of blood. Then the heart was perfused with the same solution containing 1 mg/mL collagenase Type A (Cat log #9036-06-0; Roche, Germany) and 0.08 mg/mL protease Type XIV (Cat log #48655321; Sigma-Aldrich, USA) for 8 to 11 min, followed by Tyrode solution containing 0.2 mM CaCl2 for 5 min. Single cells were then obtained by dissociation via gentle agitation of digested ventricular cardiac tissues. Ventricular cardiomyocyte suspensions were filtered through a nylon mesh, and cells were stored at room temperature in 0.2 mM CaCl2 Tyrode solution. All solutions used for dissection and perfusion were continuously bubbled with O2.

### Cardiomyocyte Contractility

Used herein was the IonOptix system (Ion Optix, Milton, MA, USA) to measure sarcomere and whole-cell contractility in enzymatically isolated ventricular myocytes from the FXN-KO and FXN-WT mice. Ventricular myocytes were placed on an IonOptix chamber and onto the stage of an inverted microscope and then perfused with 37° C. Tyrode solution containing 2 mM Ca²⁺. The cell environment was stimulated at a frequency of 1 Hz using a MyoPacer Field Stimulator (Ion Optix) connected to a pair of platinum wires placed on opposite sides of the chamber, allowing the myocytes to contract. The single cell, and sarcomere contractility analyzed using the Ionwizard software.

### Confocal Imaging

For the immunofluorescence of SERCA2a, single ventricular myocytes from both FXN-KO and FXN-WT mice were fixed in 4% formaldehyde, permeabilized in 0.5% Triton-X100, and blocked in 10% goat serum, 0.1% Tween-20 (Catalog #156054; Biorad, Hercules, CA). Primary antibodies were mouse monoclonal anti-SERCA2 (dilution 1:500; Santa Cruz Biotechnology; Catalog #sc-376235) and Alexa-546 conjugated to goat anti-mouse (Life Technologies). Cells were imaged on an Olympus Fluoview 1000 confocal microscope (Center Valley, PA).

For the measurement of ROS, ventricular myocytes were incubated with MitoSOX Red (5 μM; Invitrogen; Catalog #M36008), for 30 min at 37° C., and cells were washed with Tyrode solution. MitoSOX dye was used as a marker for ROS measurements. The live cells were imaged using Nikon 3i confocal microscope with a 63× water immersion magnification lens.

The mitochondrial membrane potential (ΔΨm) was measured using tetramethyl rhodamine methyl ester (TMRM) dye. Left ventricular myocytes from the FXN-WT and FXN-KO were plated in an eight-well confocal chamber (LAB-TEK, DK, Denmark; Catalog #155409,) for 20 minutes, and cells were incubated with TMRM (10 nmol/L; Invitrogen; Catalog #T668) for 20 minutes in Tyrode solution at 37° C. and washed two times with Tyrode solution. After incubation with the dye, live cells were imaged for 20 min in 2 min intervals on a confocal microscope (Olympus-IX) using a 63X-oil objective. After 10 minutes of baseline recording, carbonyl cyanide 4-(trifluoromethoxy) phenylhydrazone (FCCP) (10 μmol/L) was added to both FXN-WT and FXN-KO ventricular myocytes, and fluorescence was recorded for 10 more minutes. Confocal settings were identical for FXN-WT and the FXN-KO myocytes. Utilized herein was the ImageJ-FIJI (NIH, USA) software (https://fiji.sc/) to analyze the immunofluorescence of SERCA2a, ROS fluorescence, and ΔΨm.

### Oxygen Consumption Rate Mitochondrial Respiration Measurements

To investigate how mitochondrial respiration was affected in FA, OCR was measured herein using XFp24 Cell Mito Stress Test (Catalog #B05423; Agilent Seahorse). One day prior to the experiment, XFp24 cell culture plates were coated with gelatin/fibronectin at 37° C. Ventricular myocytes were enzymatically isolated from FXN-KO and FXN-WT mice. We dissociated myocytes in DMEM medium (Seahorse Bioscience), supplemented with 1 mM pyruvate, 2 mM glutamine, and 10 mM D-glucose. We plated 2500 myocytes/well into 24 well seahorse plates for 10 min. OCR were measured using the Seahorse Bioscience XF24 Extracellular Flux Analyzer (Seahorse Bioscience). Measurements were taken as the cells were incubated sequentially under four conditions: 1) basal levels were measured with no additives; 2) oligomycin (1.5 μM) was added to inhibit ATP synthase and OXPHOS, showing glycolysis alone reversibly; 3) FCCP (1 μM), a mitochondrial uncoupler, was added to induce maximal respiration; and 4) Antimycin A (10 μM), a Complex III inhibitor, was added to obtain non-mitochondrial oxygen consumption background. The Seahorse software was used to extract the results and plotted them using GraphPad Prism. OCR was normalized to cell number per well.

### Transmission Electron Microscopy (TEM)

TEM was performed as previously described. Left ventricular tissues were cut into 4 mm, washed with 0.1M sodium cacodylate, and fixed in 2.5% glutaraldehyde for 48 hours at 4° C., then transferred to sodium cacodylate containing 1% osmium tetroxide for 2 hours. Each sample was dehydrated with a graded series of ethanol (35%, 50%, 70%, and 95% in water), and 100% dry acetone was used for final dehydration for 10 minutes at room temperature. Polymerized samples were sliced with a diamond knife and transferred to copper grids. Ultrathin sections were stained with lanthanum nitrate and uranyl acetate and imaged with the JEOL JEM-1400 TEM. Low-magnification imaging was followed by high-magnification imaging (10k to 100k). Representative images were acquired and recorded with Digital Micrograph software.

### Results

### Cardiac Abnormalities in the FA Mouse

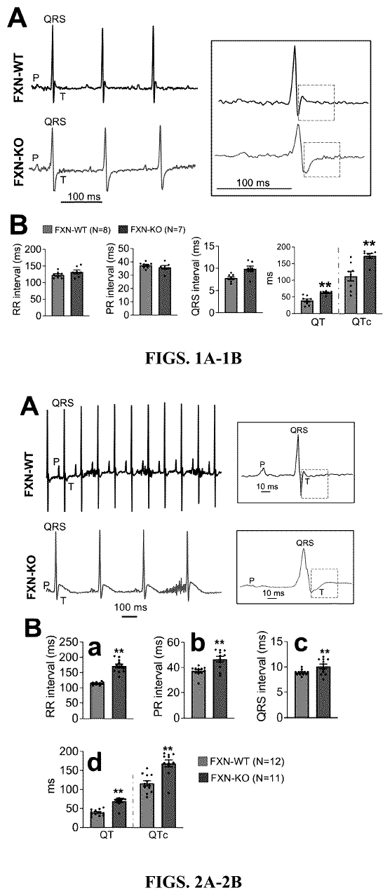

Examined herein were the ECG and echocardiographic manifestations of FA in the mouse model. Surface ECG (under 1.5% Isoflurane) was conducted in 9.5 and 12 weeks old FXN-WT and FXN-KO mice. ECG abnormalities were evident in the FXN-KO mice compared to the FXN-WT. In 9.5 weeks old animals, FXN-KO mice displayed a significant increase in QT interval duration compared to the FXN-WT (FIGS. 1A-1B). This is consistent with clinical observations where QT prolongation and T-wave inversion have been documented in patients with FA. In the 12 weeks old FXN-KO mice, the RR, PR, QRS, QT, and QTc intervals were significantly increased (FIGS. 2A, 2B; *p<0.05 and **p<0.01; unpaired 2-sample t-test).

Further assessed herein was cardiac function and morphological developments in the FXN-KO mice using echocardiography. The M-Mode images from FXN-WT and FXN-KO suggest a significantly altered cardiac function in the FXN-KO mice (FIG. 3A), where the ejection fraction and fractional shortening were significantly reduced, and the left ventricular end-diastolic and end-systolic volumes were significantly increased compared to the FXN-WT (FIG. 3B; *p<0.05 and **p<0.01; unpaired 2-sample t-test).

These results suggest that frataxin deficiency in the mouse leads to contractile dysfunction that includes a reduced ejection fraction, similar to what has been described in FA patients. Altogether, this mouse FXN-KO model (MCK-Cre) seems to recapitulate several ECG and echocardiographic manifestations that have been described in FA patients with cardiac involvement.

Frataxin deficiency alters the major Ca²⁺ handling proteins expression in the FA heart Performed herein was western blot analysis in human and mouse heart samples to explore as a step toward understanding whether FA causes, in both species, similar protein abundance changes in pathways important for intracellular Ca²⁺ handling. This is since cardiac dysfunction, specifically reduced left ventricular ejection fraction, occurs in FA patients with cardiac involvement and is shown to indicate poor prognosis.

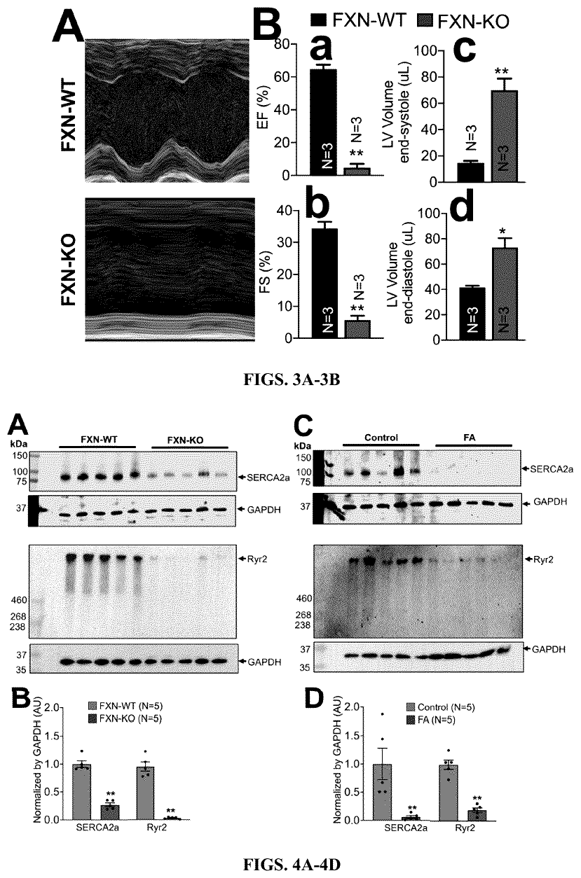

Samples of 5 FA and 5 control human ventricular samples were digested, and 5 FXN-KO and 5 control FXN-WT mouse ventricular samples, and analysis was then performed. It was discovered that SERCA2a and Ryr2 expression significantly decreased in the FA human and in the FXN-KO heart samples compared to the control or FXN-WT (FIGS. 4A-4D). In FIGS. 4A and 4C, the upper panels are representative blots of SERCA2a, and Ryr2, in mouse (FIG. 4A) and human (FIG. 4C) samples. The graphs shows the quantification of the SERCA2a, and Ryr2 proteins expression were significantly downregulated in the human and mouse FA heart samples compared to controls (**p<0.01; unpaired 2-sample t-test). Overall, these results indicate that this mouse model of FA shows proteomic changes in Ca²⁺ handling, similar to the human heart with FA.

### Cellular Phenotype Of Frataxin Deficiency in Ventricular Myocytes

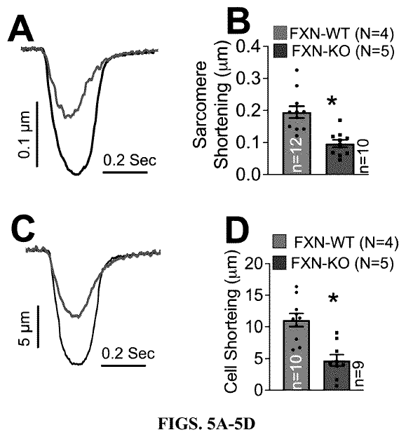

Utilized herein was the lonOptix system to investigate the cellular contractile phenotypes. Sarcomere shortening and whole cell shortening were measured (FIGS. 5A-5D). The sarcomere and whole cell shortening were significantly reduced in FXN-KO myocytes compared to FXN-WT myocytes (FIGS. 5A-5D; *p<0.05; unpaired 2-sample t-test). Further analyzed herein were the kinetics of sarcomere and whole cell contractility for the FXN-WT and FXN-KO ventricular myocytes (Table 2). For the sarcomere shortening, it was found that the magnitude of the departure and return velocities were significantly reduced, and the time to relaxation of 90% was significantly increased in FXN-KO compared to FXN-WT myocytes (Table 2; *p<0.05 and **p<0.01; unpaired 2-sample t-test). For the whole cell shortening, it was found that the return velocity was significantly reduced in FXN-KO compared to FXN-WT myocytes (Table 2; *p<0.05; unpaired 2-sample t-test).

TABLE 2
Kinetics of sarcomere and whole cell contraction
and relaxation in ventricular cardiomyocytes
from FXN-WT and KO. Data represent mean ± SEM

| | FXN-WT | FXN-KO |
|---|---|---|
| Sarcomere Shortening | | |
| Departure Velocity | −3.5 ± 0.43 | −1.77 ± 0.33 * |
| (μm/sec.) | | |
| Return velocity (μm/sec.) | 2.70 ± 0.36 | 1.10 ± 0.27 ** |
| Time to peak 90% (sec.) | 0.87 ± 0.004 | 0.10 ± 0.006 |
| Time to relaxation 90% | 0.28 ± 0.01 | 0.39 ± 0.04 * |
| (sec.) | | |
| Whole Cell Shortening | | |
| Departure Velocity | −215.66 ± 76.82 | −94.92 ± 19.12 |
| (μm/sec.) | | |
| Return velocity (μm/sec.) | 125.41 ± 35.46 | 44.99 ± 9.01 * |
| Time to peak 90% (sec.) | 0.092 ± 0.007 | 0.098 ± 0.01 |
| Time to relaxation 90% | 0.33 ± 0.05 | 0.39 ± 0.04 |
| (sec.) | | |

(* p < 0.05, ** p < 0.01, vs. FXN-WT; unpaired 2-sample t-test).

### Lack of Frataxin Triggers Mitochondrial Perturbations and Necrotic Cardiac Cell Death in FA

The transfer of electrons via electron transport chain complexes on the inner mitochondrial membrane is essential for establishing the electromotive force and proton gradient for maintaining ΔΨm. It is worth noting that mitochondrial energy production relies heavily on FeS-clusters (respiratory complexes I, II, and III) and heme (respiratory complex IV) as prosthetic groups, but the biogenesis of FeS-clusters itself takes place in mitochondria, which contain the specific assembly machinery. Frataxin was shown to interact with multiple core components of this machinery.

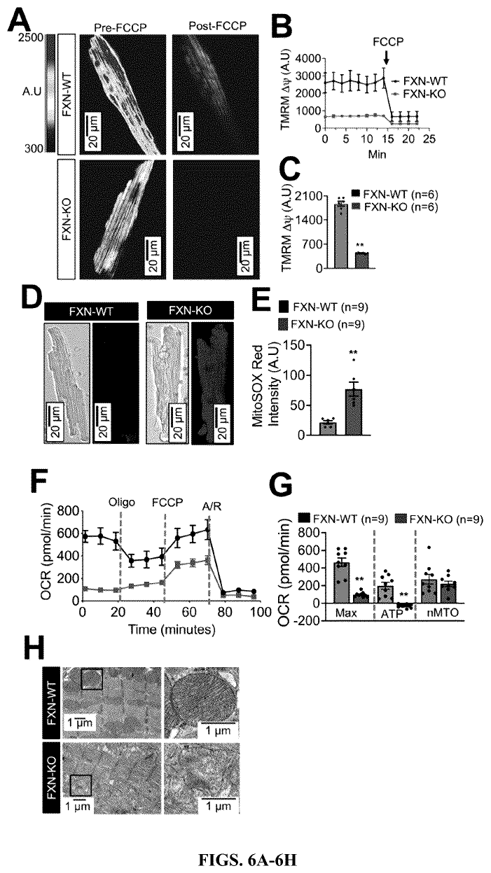

Mitochondrial function was assessed by evaluating the ΔΨm, which is a direct measurement of H⁺ ion production from proton pumps such as complex I, III, and IV in the electron transport chain during oxidative phosphorylation. The ΔΨm was significantly depolarized in FXN-KO ventricular myocytes compared to the FXN-WT (FIGS. 6A-6H). FIG. 6A shows representative microscopic images from FXN-WT (upper panel) and FXN-KO (lower panel). FIG. 6B is the time-dependent ΔΨm before and after the addition of 10 μM carbonyl cyanide 4-(trifluoromethoxy) phenylhydrazone (FCCP), a proton uncoupler that decreases and collapses the ΔΨm. FIG. 6C shows the average change in ΔΨm intensity after the addition of FCCP. The average of ΔΨm was significantly lower in FXN-KO versus FXN-WT (**p<0.01, t-test).

Tested herein was whether the observed loss of ΔΩm was accompanied by elevation of ROS in FA. FIGS. 6D and 6E show the representative fluorescence images from FXN-KO and FXN-WT. Confocal settings were identical for both conditions. FIG. 6E is the quantification of the relative fluorescence of cells with high ROS staining, which was significantly higher in FXN-KO versus control FXN-WT (**p<0.01 vs. FXN-WT, t-test, N=1; n=9 preparations each). This experiment confirmed the greater levels of ROS in FXN-KO compared to the FXN-WT.

OCR was measured to confirm that the ROS elevation might be directly related to a disruption of respiratory chain activity. XFp24 Cell Mito Stress Test (Agilent Seahorse) was used in enzymatically isolated ventricular myocytes from FXN-KO and FXN-WT mice (FIGS. 6F and 6G). Oxygen is consumed to generate ATP in the mitochondria via the oxidative phosphorylation pathway, and therefore, measuring oxygen consumption will allow for a readout of mitochondrial respiration and cellular metabolism. After measurement of basal OCR, cellular respiration modulators, oligomycin, FCCP, and antimycin A, were injected in each well in order to quantify ATP-linked respiration and maximal respiration as per the manufacturer's protocol. Oligomycin decreased OCR by inhibiting ATP synthase. FCCP, a proton uncoupler, increased OCR to maximal levels by uncoupling oxygen consumption from ATP production. Antimycin A reduced OCR by targeting the electron transport chain. FIG. 6F demonstrates the change of OCR after injection of mitochondrial modulators in ventricular myocytes from the FXN-WT and FXN-KO mice, and FIG. 6G shows that the basal respiratory rate, as well as ATP-linked respiration, was significantly reduced in FXN-KO myocytes (**p<0.01, t-test). Respiratory capacity was not changed in FXN-KO mice ventricular myocytes in contrast to FXN-WT myocytes (p =0.21, t-test).

Cardiomyocyte ultrastructure was also assessed using TEM. FXN-KO mice mitochondria displayed severe alterations with sparse cristae, compared to FXN-WT mice mitochondria. Additionally, the total volume of mitochondria in FXN-KO heart tissues appears to be greater than that in FXN-WT heart tissues in FIG. 6H. Thus, these results suggest a decreased bioenergetic efficiency and altered mitochondrial proliferation.

### Discussion

Even though FA is considered a neurodegenerative disorder, more than 50% of FA patients die from heart failure. Shown herein is that a loss in frataxin is associated with mitochondrial perturbations and cell death in ventricular myocytes likely through the dysregulation of Ca²⁺ handling proteins.

The western blotting data confirmed the significant downregulation of SERCA2a, and Ryr2 in FXN-KO mice and FA human hearts compared to controls and FXN-WT. Extensive research has been conducted, in animal models as well as in human subjects, to investigate the role of SERCA2a in heart failure. Alterations in SERCA2a expression and activity lead to a decrease in SR Ca²⁺ load and cardiac dysfunction. It has been shown that decreased SERCA2a expression may lead to cardiac hypertrophy and increased SERCA2a affinity. Decreased SERCA2a has also been known to affect Ca²⁺ signaling by slowing down the rate of decay of the systolic Ca²⁺ transient, decreasing the SR Ca²⁺ content, and thus contraction. Decreased Ryr2 is different than what has been described in classical heart failure, where Ryr2 was upregulated. Enhanced Ryr2-mediated SR Ca²⁺ leak has been observed in patients with heart failure and in various animal models, and the decrease of Ryr2 is unique in FA heart failure compared to classical heart failure.

The decreased activity of SERCA2a has effects on Ca²⁺ signaling. It slows down the rate of decay of the systolic Ca²⁺ transient, decreases the SR Ca²⁺ content, and thus contraction. In fact, in the data, the rate of contraction (departure velocity) and relaxation (return velocity) were significantly down-regulated (Table 2) in FXN-KO compared to the FXN-WT.

The ΔΨm was depolarized, and ROS levels were elevated in human induced pluripotent stem cell-derived myocytes from a patient with FA, which was confirmed in ventricular myocytes from both FXN-KO and FXN-WT. ROS levels were significantly elevated in FXN-KO ventricular myocytes compared to the FXN-WT. Also found herein was significantly decreased OCR in FXN-KO ventricular myocytes compared to the FXN-WT. In previous findings, the mitochondrial structure was disorganized in FA. In this TEM data, it was also found that disrupted myofibrils and an irregular mitochondrion were disorganized with central tubular cristae in FXN-KO ventricular myocardium compared to the FXN-WT. The increase in ROS may lead to a decrease in OCR and ΔΨm depolarization and structural changes in mitochondria. It has also been shown that decreasing expression or function of SERCA2a leads to increases in oxidative stress.

### Conclusion

Treatments for cardiac dysfunctions in FA patients remain inadequate, with beta-blockers, ACE inhibitors, and diuretics being used for the management of cardiomyopathy. Additionally, oxidative stress is increased in FA, and antioxidant supplementation has been proposed as a therapy, including Vitamin E, Coenzyme Q10, and its analog Idebenone in FA patients. These treatments do not offer protection against cardiac dysfunction progression in FA. This could be due to a lack of understanding of the EC coupling mechanism and Ca²⁺ handling in FA heart failure. Herein, it was found that major Ca²⁺ handling proteins were dysregulated in human and mouse cardiac tissue. SERCA2a and Ryr2, which are SR Ca²⁺-release and reuptake channels involved in EC coupling, were downregulated in FA. In non-FA heart failure, Ryr2 protein expression and activity are upregulated by the phosphorylation of Ryr2 via activation of CaMKII enhanced by adrenergic activity, which can potentiate SR Ca²⁺ leak and lead to arrhythmia. In contrast, decreased expression of Ryr2 was found in FA hearts.

### Example 2

### The Cardiac and Mitochondrial Calcium Handling Machinery is Remodeled in Friedreich's Ataxia

### Introduction

Ca²⁺ plays a role in the regulation of cellular function as a major intracellular messenger. Intracellular changes in Ca²⁺ concentration regulate cardiac contractility. During systole, Ca²⁺ ions enter the myocyte through L-type Ca²⁺ channels located mainly in the transverse tubules. This leads to the release of Ca²⁺ from the sarcoplasmic reticulum (SR) through the type 2 ryanodine receptors (Ryr2) in a process known as Ca²⁺-induced Ca²⁺ release. The free Ca²⁺ then binds to troponin C, which allows for the cross-bridge formation of contractile machinery and leads to muscle contraction. This free Ca²⁺ can also be taken into the mitochondria for the generation of ATP via activation of cellular processes, such as the citric acid cycle and oxidative phosphorylation. Ca²⁺ transport to and from the mitochondria is tightly regulated by several mitochondrial membrane proteins, including voltage-dependent anion channel (VDAC), mitochondrial Ca²⁺ uniport (MCU), and the mitochondrial sodium-Ca²⁺ exchanger (NCLX).

During diastole, Ca²⁺ is then removed from the cytoplasm for relaxation to occur, where Ca²⁺ is pumped back into the SR by the SERCA2a and extruded out of the cell through the sodium-Ca²⁺ exchanger (NCX). Phasoplamban is the regulatory protein of SERCA2a. Phosphorylation of PLN (p-PLN) can occur at three distinct sites. Ser 16 is activated by cyclic AMP and GMP (PKA and PKG), and Thr17 is activated by CaMKII. Phosphorylation of these sites inhibits the PLN association with SERCA2a, leading to increased rates of SR Ca²⁺ uptake, accelerates relaxation, and enhances SR Ca²⁺ load. PLN can be in pentameric or monomeric configurations. However, the monomeric form is more crucial since it has a higher affinity for interacting with SERCA2a.

Alterations or mutations in Ca²⁺ handling proteins, such as Ryr2, SERCA2a, and NCX, can result in cardiac pathology and arrhythmias. Additionally, irregularities in mitochondrial machinery can be detrimental to cardiomyocyte survival, resulting in progression to heart failure.

Investigated herein is whether the Ca²⁺ handling machinery is remodeled in FA. It was found that Ca²⁺ handling was dysregulated, and cardiac contractility and cellular function were decreased.

### Methods

### FA Animal Model

Used herein was the FA mouse model B6.Cg-Fxn^{em2Lutzy} Fxn^{em2.1Lutzy}Tg(Ckmm-cre)5Khn/J (FXN-KO) and littermates control Heterozygous for Fxn^{em2Lutzy}, Wildtype for Fxn^{em2.1Lutzy}, Hemizygous for Tg(Ckmm-cre)5Khn (FXN-WT) purchased from the Jackson Laboratory (Stock #029720). Also generated herein were FXN-KO and FXN-WT mice (Stock #029720) by breeding male C57BL/6J-congenic Fxn^{em2Lutzy} (homozygous frataxin floxed exon 2) (Stock #028520) and female C57BL/6-congenic Fxn^{em2.1Lutzy} Tg (Ckmm-cre)5Khn (heterozygous frataxin global knockout and homozygous MCK-Cre) (Stock #029100), and these breeders were purchased from the Jackson Laboratory. For euthanasia, CO2 was used to induce loss of consciousness, and after the loss of the pinch reflex, swift cervical dislocation was performed, followed by rapid harvesting of the heart via thoracotomy. This method is consistent with the guidelines of the American Veterinary Medical Association.

### Human Ventricular Tissues

Left ventricular samples were obtained from five different decedent donors with FA from Friedreich's Ataxia Research Alliance (FARA) tissue bank at Albany VA Hospital. I also obtained left ventricular samples from five age- and sex-matched samples from donors with no diagnosis of FA. These control hearts were rejected for transplant and obtained through a USF agreement with Lifelink®, an FDA-approved, Tampa-based organization for organ procurement (listed in Table 1).

### Proteomics

Proteomic analysis was conducted in human left ventricular samples from five different donors with FA and five age, and sex-matched samples from donors without an FA diagnosis (Table 1). Also conducted herein were proteomics in three FXN-KO and FXN-WT mice. Samples were lysed in RIPA buffer and 1X protease inhibitor cocktail (catalog #87785ThermoFisher). SDS (5% final concentration) was added to 50 μg of the protein samples. All samples were prepped with S-Trap micro columns (catalog Protifi) according to the manufacturer's protocol. For LC-MS analysis, the peptides were characterized using a Thermo Q-exactive-HF-X mass spectrometer coupled to a Thermo Easy nLC 1200. Samples were separated at 300 nL/min on an Acclaim PEPMAP 100 trap (75 μm, 2 cm, c18 3 um, 100A) and a Thermo easy spray Column (75 μm, 25 cm, c18, 100A) using a standard 120-min gradient with an initial starting condition of 2% buffer B (0.1% formic acid in 90% Acetonitrile) and 98% buffer A (0.1% formic acid in water). The mass spectrometer was outfitted with a Thermo nanospray easy source with the following parameters: Spray voltage: 1.8, Capillary temperature: 250dC, Funnel RF level=40. Parameters for data acquisition were as follows: for MS data the resolution was 60,000 with an AGC target of 3×10⁶ and a max IT time of 50 ms, the range was set to 400-1600 m/z. MS/MS data were acquired with a resolution of 15,000, an AGC of 1×10⁵, max IT of 50 ms, and the top 30 peaks were picked with an isolation window of 1.6 m/z with a dynamic execution of 25 s. Data were processed using Max quant 2.0.3.1 free software. A reviewed mouse database was downloaded from Uniprot and searched with the following parameters: a precursor mass tolerance of 10 ppm, and a fragment mass tolerance of 0.02 Da. FDR rate was set at 0.01, LFQ intensities were compared between samples for ID. LFQ intensities of proteins detected above the threshold for at least two out of three samples for each sample group were then assessed for differential abundance. Abundance ratios were then normalized across all proteins such that 0 indicated no difference between the sexes, and the largest number indicated greatest difference. Proteins having a Z score>1 and Welch's t-test p-value<0.05 were considered significant. The differentially abundant proteins were assessed for gene ontology enrichment against a background of all proteins detected above the threshold for which the FXN-KO/WT or FA/control ratio could be determined. Gene ontology categories with a p-value<0.05 were considered enriched.

### Western Blotting

For collecting lysates from the left ventricle, samples from FXN-KO, FXN-WT, human FA and control ventricular tissues were flash frozen in liquid nitrogen, and tissues were cryo-powdered with a Bessman tissue pulverizer (Spectrum Med, San Diego, CA, USA). Protein isolation was completed by adding 10 μL/mg of a RIPA lysis buffer to the cryopowder. The total lysate was centrifuged at 12,000 rpm for 20 min, and the supernatant was collected. Protein concentrations were determined with a BCA assay (Pierce™ BCA Protein Assay Kit; #23225, ThermoFisher, Carlsbad, CA, USA). Total protein (5˜15 μg) was loaded and separated by 4-20% pre-made SDS-PAGE gels (#4568095, BioRad, Mini-PROTEAN TGX Stain-Free Precast Gels, Irvine, CA, USA), then transferred to a nitrocellulose membrane, and probed with the following primary antibodies: mouse monoclonal anti-Frataxin (dilution 5 μg/ml; Abcam; Catalog #ab110328), and mouse monoclonal anti-Ryr2 (dilution 1:1000; Santa Cruz Biotechnology; Catalog #sc-376507), mouse monoclonal anti-SERCA2a (dilution 1:500; Santa Cruz Biotechnology; Catalog #sc-376235). PLN (dilution 1:1000; Abcam; Catalog #ab126174), rabbit polyclonal p-PLN at Ser 16 (dilution 0.5-2 μg/ml; Abcam; Catalog #ab15000), and p-PLN at Thr 17 Rabbit (dilution 1:400; ABclonal; Catalog #AP0910). The following secondary antibodies were used: anti-mouse IgGk BP-HRP (dilution 1:500; Santa Cruz Biotechnology; Catalog #sc-516102) for detecting Ryr2, fluorescent IRDye 680 goat anti-mouse antibodies (1:25,000; LICOR; Catalog #925-68072) for SERCA2a for 1 hr. at room temperature. The final images were detected with LiCOR Odyssey, and chemo doc and protein bands were quantified using Image Studio-LiCOR, software-2019, and FIJI-ImageJ.

### Single-Cell Isolation

Ventricular myocytes were enzymatically dissociated from young and old WT and KO mice. In short, immediately after cardiac excision, the heart was cleaned, and the aorta was cannulated. The heart was then retrogradely perfused at 2 mL/min at 36±0.5° C., for 3 min with Ca²⁺ free Tyrode solution (in mM): NaCl 137, KCl 5.4, HEPES 10, MgCl₂ 1, and glucose 10 (pH 7.3) until the effluent was clear of blood. Then the heart was perfused with the same solution containing 1 mg/mL collagenase Type A (Catalog #9036-06-0; Roche, Germany) and 0.08 mg/mL protease Type XIV (Cat log #48655321; Sigma-Aldrich, USA) for 8 to 11 min, followed by Tyrode solution containing 0.2 mM CaCl2 for 5 min. Single cells were then obtained by dissociation via gentle agitation of digested ventricular cardiac tissues. Ventricular cardiomyocyte suspensions were filtered through a nylon mesh, and cells were stored at room temperature in 0.2 mM CaCl₂ Tyrode solution. All solutions used for dissection and perfusion were continuously bubbled with O₂.

### Cardiomyocyte Contractility

The IonOptix system (Ion Optix, Milton, MA, USA) was used to measure sarcomere and whole-cell contractility in enzymatically isolated ventricular myocytes from the FXN-KO and FXN-WT mice. Ventricular myocytes were placed on an IonOptix chamber and onto the stage of an inverted microscope and then perfused with 37° C. Tyrode solution containing 2 mM Ca²⁺. Cells were stimulated at a frequency of 1 Hz using a MyoPacer Field Stimulator (Ion Optix) connected to a pair of platinum wires placed on opposite sides of the chamber. The single cell and sarcomere contractility were analyzed using the Ionwizard software.

### Results

### Proteomics Analysis Shows Extensive Alteration in Cardiac Contractile Proteins Expression in FA Human and Mouse Hearts

Proteomics analysis was conducted in human and mouse heart samples to explore as a step toward understanding whether FA causes, in both species, similar protein abundance changes in pathways important for intracellular Ca²⁺ handling. This is because cardiac dysfunction, specifically reduced left ventricular ejection fraction, occurs in FA patients with cardiac involvement and is shown to indicate poor prognosis. LC-MS was conducted on the digested samples of 5 FA and 5 control human ventricular samples, and 3 FXN-KO and 3 control FXN-WT mouse ventricular samples, and analysis was then performed. Found herein was significantly up and down-regulated cardiac contractile and Ca²⁺ handling proteins in the FA versus control human and mouse samples (FIG. 7A). Also discussed herein is significantly decreased SERCA2a, Ryr2, and Ca²⁺/calmodulin-dependent protein kinase II (CaMKII) in both mouse and human FA samples. Based on KEGG analysis, it was shown that the differentially regulated proteins using the KEGG cardiac muscle contraction pathway illustration with the dark grey denoting human and light grey marks denoting mouse (FIG. 7B).

### Frataxin Deficiency Alters the Major Ca²⁺ Handling Proteins Expression in the FA Heart

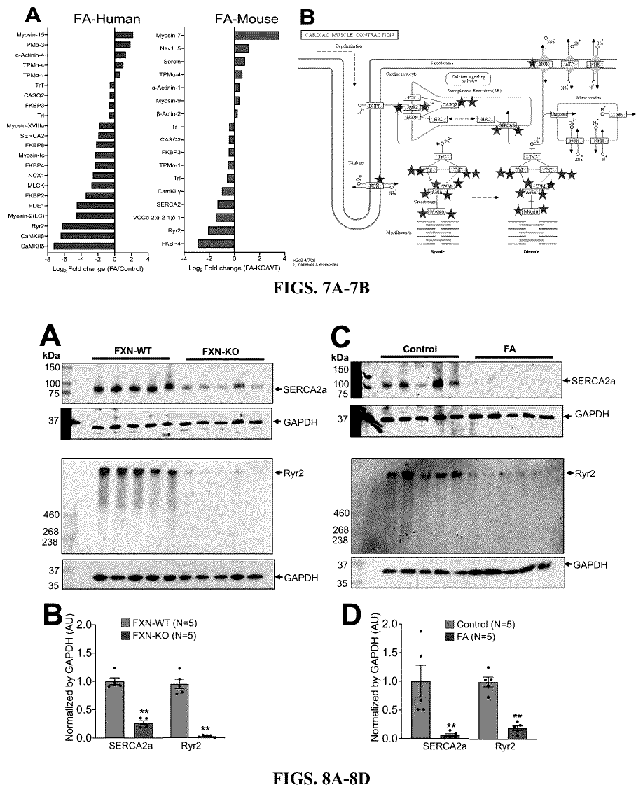

Then it was confirmed with western blot the proteomics results that SERCA2a and Ryr2 are decreased in the FA human heart samples and in the mouse model. In FIGS. 8A-8D, the upper panels are representative blots of SERCA2a and Ryr2 in mouse (FIGS. 8A and 8B) and human (FIGS. 8C and 8D) samples. The graphs quantify the protein expression, where SERCA2a and Ryr2 proteins were significantly downregulated in the human and mouse FA heart samples compared to controls (**p<0.01; unpaired 2-sample t-test), lending support to the proteomics analysis. Overall, these results indicate that this mouse model of FA shows proteomic changes in Ca²⁺ handling, similar to the human heart with FA.

In heart failure, the decrease in SERCA2a expression is accompanied by a smaller decrease or no change in PLN expression, resulting in a decrease in the SERCA2a/PLN ratio and a further decrease in SR Ca²⁺ uptake. Investigated herein is whether the expression of PLN and p-PLN were remodeled in FA. In the data, significantly decreased protein expression of PLN and p-PLN at Ser 16 and Thr 17 in the lysates from FXN-KO was found compared to the FXN-WT (FIGS. 9A-9D; **p<0.01; unpaired 2-sample t-test). In FA human hearts, the total PLN was decreased, but it was not statistically significant compared to the control. The decrease in total PLN was only observed in young FA hearts, and there was no change in old FA human hearts (FIGS. 9A-9D and the age and gender of human samples are listed in Table 1).

During systole, cytosolic Ca²⁺ rises throughout the cell, and a portion of the increased cytosolic Ca²⁺ enters the mitochondrial matrix, where it activates ATP production for cellular function. In order to maintain the optimum amount of Ca²⁺ for ATP production in mitochondria, Ca²⁺ influx must be balanced by Ca²⁺ efflux. Ca²⁺ enters the mitochondrial matrix via the MCU and exits via the NCLX. The VDACs serve as a mitochondrial gatekeeper, mediating the fluxes of ions, nucleotides, and other metabolites across the outer mitochondrial membrane and controlling the metabolic and energy crosstalk between mitochondria and the rest of the cell. VDACs also regulate the transport of Ca²⁺ in and out of the mitochondria. To investigate the mitochondrial Ca²⁺ regulatory proteins expression in FA, western blots were performed, and in FIGS. 7A-7B, it was found that the expression of VDAC and MCU proteins were significantly higher in the FXN-KO mice versus FXN-WT (*p<0.05, t-test). There was no change in NLCX protein expression in the FXN-KO mice compared to the FXN-WT. These results are in line with studies showing that VDAC and MCU protein expression was elevated in patients with HF.

The cellular Contractile Phenotype of Frataxin Deficiency in Ventricular Myocytes

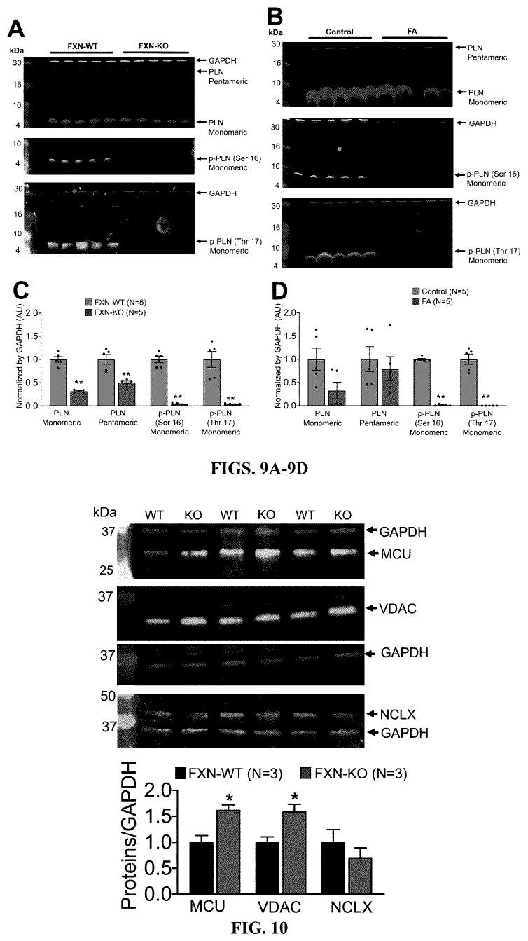

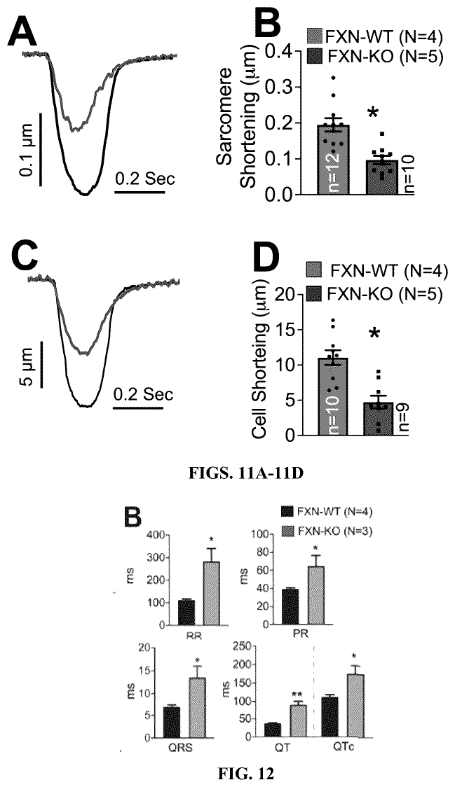

Utilized herein is the IonOptix system to measure sarcomere shortening and whole cell shortening (FIGS. 11A-11D). The sarcomere and whole cell shortening were significantly reduced in FXN-KO myocytes compared to FXN-WT myocytes (FIG. 10; *p<0.05; unpaired 2-sample t-test). Further analyzed herein the kinetics of sarcomere and whole cell contractility for the FXN-WT and FXN-KO ventricular myocytes (Table 2). For the sarcomere shortening, it was found that the magnitude of the departure and return velocities were significantly reduced, and the time to relaxation of 90% was significantly increased in FXN-KO compared to FXN-WT myocytes (Table 2; *p<0.05 and **p<0.01; unpaired 2-sample 1-test). For the whole cell shortening, it was found that the return velocity was significantly reduced in FXN-KO compared to FXN-WT myocytes (Table 2; *p<0.05; unpaired 2-sample t-test).

### Phasoplamban Upregulation

Furthermore, it was found that the total PLN protein expression was significantly decreased in FXN-KO left ventricle tissues compared to FXN-WT, but the total PLN was drastically decreased in less than 30 years of FA patient's left ventricle tissue compared to the old FA patient's left ventricle tissues (Table 1).

### Discussion

Even though FA is considered a neurodegenerative disorder, more than 50% of FA patients die from heart failure. In this study, it was shown that a loss in frataxin is associated with mitochondrial perturbations and cell death in ventricular myocytes through the dysregulation of Ca²⁺ handling proteins. The western blotting data confirmed the significant downregulation of SERCA2a, and Ryr2 in FXN-KO mice and FA human hearts compared to respective controls. Extensive research has been conducted in animal models as well as in human subjects to investigate the role of SERCA2a in heart failure. Alterations in SERCA2a expression and activity lead to a decrease in SR Ca²⁺ load and cardiac dysfunction. Decreased SERCA2a has also been known to affect Ca²⁺ signaling by slowing down the rate of decay of the systolic Ca²⁺ transient, decreasing the SR Ca²⁺ content, and thus contraction.

Decreased Ryr2 is different than what has been described in classical heart failure, where Ryr2 was upregulated. Enhanced Ryr2-mediated SR Ca²⁺ leak has been observed in patients with heart failure and in various animal models, and the decrease of Ryr2 is unique in FA heart failure compared to classical heart failure.

The decreased activity of SERCA2a has effects on Ca²⁺ signaling. It slows down the rate of decay of the systolic Ca²⁺ transient, decreases the SR Ca²⁺ content, and thus contraction. In fact, the rate of contraction (departure velocity) and relaxation (return velocity) were significantly down-regulated (Table 2) in FXN-KO compared to the FXN-WT.

In heart failure, the decrease in SERCA2a expression is accompanied by a smaller decrease or no change in PLN expression, resulting in a decrease in the SERCA2a/PLN ratio and a further decrease in SR Ca²⁺ uptake. In the data, the expression of PLN and p-PLN at ASer16 and Thr17 significantly decreased in FA and FXN-KO hearts compared to the control and FXN-WT. However, the total PLN levels did not change in FA hearts aged above 65 years old compared to the young and control. The decrease of p-PLN protein expression has been shown in cardiomyopathy or heart failure. In FA, the decreased SERCA2a and p-PLN levels lead to elevated diastolic calcium. This can result in decreased CaMKII activity. Moreover, in the proteomics data, CaMKII levels seem to be significantly decreased in both FA human and FXN-KO mouse hearts compared to the control or FXN-WT.

Abnormal cytosolic Ca²⁺ causes mitochondrial Ca²⁺ overload and dysfunction in HF. Mostly, MCU may mediate acute mitochondrial Ca²⁺ overload and induce cardiomyocyte death. VDAC levels were reported to be increased in the left ventricular tissues of patients with hypertrophic cardiomyopathy. MCU and VDAC significantly increased in FA human and mouse hearts compared to the controls.

### Conclusion

Treatments for cardiac dysfunctions in FA patients remain inadequate, with beta-blockers, ACE inhibitors, and diuretics being used for the management of cardiomyopathy. Additionally, oxidative stress is increased in FA, and antioxidant supplementation has been proposed as a therapy, including Vitamin E, Coenzyme Q10, and its analog Idebenone in FA patients. These treatments do not offer protection against cardiac dysfunction progression in FA. This could be due to a lack of understanding of the EC coupling mechanism and Ca²⁺ handling in FA heart failure. Investigated herein is the EC coupling mechanism and Ca²⁺ handling in FA hearts, and it was found that major Ca²⁺ handling proteins were dysregulated in human and mouse cardiac tissue.

Importantly, SERCA2a and Ryr2, which are SR Ca²⁺-release and reuptake channels involved in EC coupling, were downregulated in FA. In non-FA heart failure, Ryr2 protein expression and activity are upregulated by the phosphorylation of Ryr2 via activation of CaMKII enhanced by adrenergic activity, which can potentiate SR Ca²⁺ leak and lead to arrhythmia. In contrast, it was found that decreased expression of Ryr2 in FA hearts. Discussed herein is that the total PLN protein expression was significantly decreased in FXN-KO left ventricle tissues compared to FXN-WT, but the total PLN was drastically decreased in less than 30 years of FA patient's left ventricle tissue compared to the old FA patient's left ventricle tissues (Table 1).

FA affects both children (early-onset) and adults (late-onset), and the mean age of early-onset FA is 10.52 years, and the mean age of death is 37.54 years. The mean age of late-onset FA is 24.4 years, and the mean age of death is 75 years. In a few rare cases, FA can be detected after 40 years old, and some patients are not diagnosed during life. Early-onset FA leads to cardiac abnormalities and cardiomyopathy fatality, but patients with late-onset FA have no cardiomyopathy; they die from disabling ataxia or illnesses that are unrelated to FA. It was found that the SERCA2a and Ryr2 were significantly decreased in both young and old FA patient's heart samples, but the total PLN decreased only in young FA patient's hearts. The decrease in total PLN could be a possible mechanism for developing cardiac abnormalities in young FA patients (early-onset).

In summary, the major Ca²⁺ handling proteins SERCA2a, Ryr2, and PLN were significantly decreased, and the major mitochondrial Ca²⁺ handling proteins MCU and VDAC were significantly upregulated in FA. It was established that Ca²⁺ handling proteins SERCA2a, Ryr2, and PLN could possibly renormalize calcium handling, contractility, and mitochondrial function, leading to the mitigation of cardiac contractile dysfunction in FA.

### Example 3

### Dysregulation of Proteins in Friedrich's Ataxia

### Background

Friedrich's ataxia (FA) is an inherited neurodegenerative disorder that causes progressive nervous system damage resulting in impaired muscle coordination. FA is caused by an expansion of the DNA triplet guanine-adenine-adenine (GAA) in the first intron of the Frataxin gene (FXN), located on chromosome 9q13. In the unaffected population, the number of GAA repeats range from 6 to 27. In FA patients, the number of GAA repeats ranges from 44 to 1,700, which decrease frataxin protein expression. Although FA is considered a neurodegenerative disorder, 59% of FA patients die due to heart disease.

The objective of the data herein was to determine whether an abnormal calcium handling machinery is a molecular mechanism that could perpetuate cardiac dysfunction in FA.

### Methods

### Proteomics Analysis

Proteomic analysis was performed on 1) left ventricular samples from a frataxin KO (FXN-KO) mouse model vs WT, and 2) left ventricular tissue biopsies from decedent patients with FA vs. ventricular tissue from no FA human hearts rejected for transplant and obtained from the Lifelink®. Protein lysates were analyzed using LC-MS and characterized using a Thermo Q-executive-HF-X mass spectrometer. LFQ intensities were compared between samples.

### Western Blot

Protein samples were separated by SDS-PAGE. Membranes were probed with VDAC, MCU, and GAPDH antibodies.

### Echocardiography

Transthoracic echocardiography was performed using a Vevo3100LT (VisualSonics) equipped with a 30 MHz transducer.

### ECG

ECG was measured using animal Bio Amp and PowerLab 4/35 from AD instruments.

Confocal Imaging

The mitochondrial membrane potential (ΔΨm) and mitochondrial ROS were quantified with TMRM and mitoSOX red dyes respectively in isolated ventricular myocytes.

### OCR

Oxygen consumption rate (OCR) was measured with the Agilent Seahorse.

### Results

### FIGS. 7A-7B

Proteomics analysis in mouse and human hearts showed significantly upregulated and downregulated proteins in human hearts with FA (N=5) compared to control (N=5) and in FXN-KO mouse hearts (N=3) versus WT (N=3).

### FIGS. 2A-2B, FIG. 12

Abnormal ECG in FXN-KO mice showed representative ECG tracings for FXN-WT and KO mice and summary data comparing the duration of RR, PR, QRS, QT, and QTc intervals from WT vs. KO mice.

This data was the initial findings in several animals. This ECG experiment was later repeated in several animals at varying ages and a difference was discovered in ECG from the 9.5 weeks old FXN-KO animals (FIGS. 1A-1B). Around 12.5 weeks olfactory, the FXN-KO animals underwent significant changes in several ECG parameters.

### FIGS. 3A-3B

Echocardiography M-mode images and summary bar graphs of (a) left ventricular ejection fraction (EF), (b) fractional shortening (FS), (c) left ventricular end-systolic volume, and (d) left ventricular end-diastolic volume.

### FIGS. 6A-6C

TMRM mitochondrial membrane potential (ΔΨm) shows (A) visualization of FXN-KO and WT fluorescence and (B) time-dependent ΔΨm over 22 min. After 15 minutes, the addition of 10 μm FCCP decreased ΔΨm. The ΔΨm was significantly reduced in FXN-KO.

### FIG. 10

Mitochondrial Ca²⁺ gating proteins expression in KO vs. WT showed representative blots of MCU, VDAC, and GAPDH and bar graphs show that MCU and VDAC were significantly changed in KO mice, compared to the WT.

### FIGS. 6D-6E

Elevated ROS levels in F showed MitoSOX superoxide indicator reveals greater ROS level in FXN-KO ventricular myocytes compared to WT and data represent the ROS fluorescence was significantly higher in KO.

### FIGS. 6F-6G

Oxygen consumption rate (OCR) in FXN-KO and WT relates to a curve shows the change in OCR after injections of oligomycin, FCCP, Antimycin, and Rotenone and quantification of normalized maximal OCR (max), ATP-linked OCR (ATP), and nonmitochondrial (nMTO).

Conclusion Several contractile proteins were dysregulated in FA. MCU and VDAC are upregulated, which could subsequently result in ΔΨm depolarization and ROS generation, via mitochondrial Ca²⁺ overload. Remodeling of cardiac Ca²⁺ signaling could be the major cause of cardiac dysfunction in FA.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### REFERENCES

Andersson DC, Fauconnier J, Yamada T, Lacampagne A, Zhang SJ, Katz A and Westerblad H. Mitochondrial production of reactive oxygen species contributes to the β-adrenergic stimulation of mouse cardiomycytes. J Physiol. 2011;589:1791-801.

Au-Tachibana S, Au-Chen C, Au-Zhang OR, Au-Schurr SV, Au-Hill C, Au-Li R, Au-Manso AM, Au-Zhang J, Au-Andreyev A, Au-Murphy AN, Au-Ross RS and Au-Cho Y. Analyzing Oxygen Consumption Rate in Primary Cultured Mouse Neonatal Cardiomyocytes Using an Extracellular Flux Analyzer. JoVE. 2019: e59052

Belevych A, Kubalova Z, Terentyev D, Hamlin RL, Carnes CA and Györke S. Enhanced ryanodine receptor-mediated calcium leak determines reduced sarcoplasmic reticulum calcium content in chronic canine heart failure. Biophysical journal. 2007;93:4083-92.

Bers DM. Ryanodine Receptor S2808 Phosphorylation in Heart Failure. Circulation research. 2012; 110:796-799.

Brown DA, Perry JB, Allen ME, Sabbah HN, Stauffer BL, Shaikh SR, Cleland JGF, Colucci WS, Butler J, Voors AA, Anker SD, Pitt B, Pieske B, Filippatos G, Greene SJ and Gheorghiade M. Mitochondrial function as a therapeutic target in heart failure. Nature Reviews Cardiology. 2017; 14:238-250.

Bulteau AL, O'Neill HA, Kennedy MC, Ikeda-Saito M, Isaya G and Szweda LI. Frataxin acts as an iron chaperone protein to modulate mitochondrial aconitase activity. Science. 2004;305:242-5.

Campuzano V, Montermini L, Molto MD, Pianese L, Cossee M, Cavalcanti F, Monros E, Rodius F, Duclos F, Monticelli A, Zara F, Canizares J, Koutnikova H, Bidichandani SI, Gellera C, Brice A, Trouillas P, De Michele G, Filla A, De Frutos R, Palau F, Patel PI, Di Donato S, Mandel JL, Cocozza S, Koenig M and Pandolfo M. Friedreich's ataxia: autosomal recessive disease caused by an intronic GAA triplet repeat expansion. Science. 1996;271:1423-7.Pandolfo M. Molecular pathogenesis of Friedreich ataxia. Arch Neurol. 1999;56:1201-8.

Carafoli E and Lehninger AL. A survey of the interaction of calcium ions with mitochondria from different tissues and species. Biochem J. 1971; 122:681-90.

Chalmers S and McCarron JG. The mitochondrial membrane potential and Ca&lt;sup&gt;2+&lt;/sup&gt; oscillations in smooth muscle. Journal of Cell Science. 2008; 121:75.

Chhabra L, Devadoss R, Chaubey VK and Spodick DH. Interatrial block in the modern era. Curr Cardiol Rev. 2014; 10:181-9.

Chidipi B, Angulo MB, Shah SI, Rieser M, Ullah G, McDonald TV and Noujaim SF. The dynamin-related protein 1 is decreased and the mitochondrial network is altered in Friedreich's ataxia cardiomyopathy. Int J Biochem Cell Biol. 2022; 143:106137.

Chidipi B, Shah SI, Reiser M, Kanithi M, Garces A, Cha BJ, Ullah G and Noujaim SF. All-Trans Retinoic Acid Increases DRP1 Levels and Promotes Mitochondrial Fission. Cells. 2021;10.

Child JS, Perloff JK, Bach PM, Wolfe AD, Perlman S and Kark RA. Cardiac involvement in Friedreich's ataxia: a clinical study of 75 patients. J Am Coll Cardiol. 1986;7:1370-8.

Chu G and Kranias EG. Phospholamban as a Therapeutic Modality in Heart Failure Heart Failure: Molecules, Mechanisms and Therapeutic Targets; 2006:156-175.

De Michele G, Filla A, Barbieri F, Perretti A, Santoro L, Trombetta L, Santorelli F and Campanella G. Late onset recessive ataxia with Friedreich's disease phenotype. J Neurol Neurosurg Psychiatry. 1989;52:1398-401.

Delatycki MB and Corben LA. Clinical features of Friedreich ataxia. J Child Neurol. 2012;27:1133-7.

Dematteis G, Vydmantaitė G, Ruffinatti FA, Chahin M, Farruggio S, Barberis E, Ferrari E, Marengo E, Distasi C, Morkūnienė R, Genazzani AA, Grilli M, Grossini E, Corazzari M, Manfredi M, Lim D, Jekabsone A and Tapella L. Proteomic analysis links alterations of bioenergetics, mitochondria-ER interactions and proteostasis in hippocampal astrocytes from 3xTg-AD mice. Cell Death & Disease. 2020; 11:645

Denton RM and McCormack JG. The role of calcium in the regulation of mitochondrial metabolism. Biochemical Society transactions. 1980;8:266-8.

Dobrev D and Wehrens XHT. Role of RyR2 Phosphorylation in Heart Failure and Arrhythmias. Circulation research. 2014; 114:1311-1319.

Dürr A, Cossee M, Agid Y, Campuzano V, Mignard C, Penet C, Mandel JL, Brice A and Koenig M. Clinical and genetic abnormalities in patients with Friedreich's ataxia. The New England journal of medicine. 1996;335:1169-75.

Eisner D, Caldwell J and Trafford A. Sarcoplasmic Reticulum Ca-ATPase and Heart Failure 20 Years Later. Circulation research. 2013;113:958-961.

Giugliano GR and Sethi PS. Friedreich's ataxia as a cause of premature coronary artery disease. Tex Heart Inst J. 2007;34:214-7.

Goh KY, Qu J, Hong H, Liu T, Dell'Italia LJ, Wu Y, O'Rourke B and Zhou L. Impaired mitochondrial network excitability in failing guinea-pig cardiomyocytes. Cardiovascular research. 2015;109:79-89.

Hanson E, Sheldon M, Pacheco B, Alkubeysi M and Raizada V. Heart disease in Friedreich's ataxia. World J Cardiol. 2019; 11:1-12.

HARDING AE and HEWER RL. The Heart Disease of Friedreich's Ataxia: a Clinical and Electrocardiogramady of 115 Patients, with an Analysis of Serial Electrocardiogramanges in 30 Cases. QJM: An International Journal of Medicine. 1983;52:489-502.

Harding AE. FRIEDREICH'S ATAXIA: A CLINICAL AND GENETIC STUDY OF 90 FAMILIES WITH AN ANALYSIS OF EARLY DIAGNOSTIC CRITERIA AND INTRAFAMILIAL CLUSTERING OF CLINICAL FEATURES. Brain. 1981;104:589-620.

Hasenfuss G, Meyer M, Schillinger W, Preuss M, Pieske B and Just H. Calcium handling proteins in the failing human heart. Basic research in cardiology. 1997;92 Suppl 1:87-93.

Kawase Y and Hajjar RJ. The cardiac sarcoplasmic/endoplasmic reticulum calcium ATPase: a potent target for cardiovascular diseases. Nat Clin Pract Cardiovasc Med. 2008;5:554-65.

Keefe JA, Moore OM, Ho KS and Wehrens XHT. Role of Ca (2+) in healthy and pathologic cardiac function: from normal excitation-contraction coupling to mutations that cause inherited arrhythmia. Arch Toxicol. 2023;97:73-92.

Kimura Y, Kurzydlowski K, Tada M and MacLennan DH. Phospholamban Inhibitory Function Is Activated by Depolymerization*. Journal of Biological Chemistry. 1997;272:15061-15064.

Kirchberger MA, Tada M, Repke DI and Katz AM. Cyclic adenosine 3′,5′-monophosphate-dependent protein kinase stimulation of calcium uptake by canine cardiac microsomes. Journal of molecular and cellular cardiology. 1972;4:673-680.

Knowlton AA, Chen L and Malik ZA. Heart failure and mitochondrial dysfunction: the role of mitochondrial fission/fusion abnormalities and new therapeutic strategies. Journal of cardiovascular pharmacology. 2014;63:196-206.

Koeppen A, Manto M and Pandolfo M. The cerebellum and its disorders. 2002.

Koeppen AH, Morral JA, McComb RD and Feustel PJ. The neuropathology of late-onset Friedreich's ataxia. Cerebellum. 2011; 10:96-103.

Kwong JQ, Lu X, Correll RN, Schwanekamp JA, Vagnozzi RJ, Sargent MA, York AJ, Zhang J, Bers DM and Molkentin JD. The Mitochondrial Calcium Uniporter Selectively Matches Metabolic Output to Acute Contractile Stress in the Heart. Cell Rep. 2015;12:15-22.

Lee JM, Turner I, Agarwal A and Fynn SP. An unusual atrial tachycardia in a patient with Friedreich ataxia. Europace: European pacing, arrhythmias, and cardiac electrophysiology: journal of the working groups on cardiac pacing, arrhythmias, and cardiac cellular electrophysiology of the European Society of Cardiology. 2011;13:1660-1.

Legrand L, Diallo A, Monin ML, Ewenczyk C, Charles P, Isnard R, Vicaut E, Montalescot G, Durr A and Pousset F. Predictors of Left Ventricular Dysfunction in Friedreich's Ataxia in a 16-Year Observational Study. Am J Cardiovasc Drugs. 2020;20:209-216.

Li C, Ma Q, Toan S, Wang J, Zhou H and Liang J. SERCA overexpression reduces reperfusion-mediated cardiac microvascular damage through inhibition of the calcium/MCU/mPTP/necroptosis signaling pathways. Redox Biol. 2020;36:101659.

Lipskaia L, Ly H, Kawase Y, Hajjar RJ and Lompre AM. Treatment of heart failure by calcium cycling gene therapy. Future cardiology. 2007;3:413-23.

Lodi R, Tonon C, Calabrese V and Schapira AH. Friedreich's ataxia: from disease mechanisms to therapeutic interventions. Antioxid Redox Signal. 2006;8:438-43.

Luo M and Anderson ME. Mechanisms of Altered Ca<sup>2+</sup> Handling in Heart Failure. Circulation research. 2013; 113:690-708.

MacLennan DH and Kranias EG. Phospholamban: a crucial regulator of cardiac contractility. Nature Reviews Molecular Cell Biology. 2003;4:566-577.

Madesh M and Hajnóczky Gr. VDAC-dependent permeabilization of the outer mitochondrial membrane by superoxide induces rapid and massive cytochrome c release. The Journal of cell biology. 2001; 155:1003-1016.

Mariotti C, Solari A, Torta D, Marano L, Fiorentini C and Di Donato S. Idebenone treatment in Friedreich patients: One-year-long randomized placebo-controlled trial. Neurology. 2003;60:1676.

Mastroianno S, Germano M, Maggio A, Massaro R, Potenza DR, Russo A, Carella M and Di Stolfo G. Electrocardiogram in Friedreich's ataxia: A short-term surrogate endpoint for treatment efficacy. Ann Noninvasive Electrocardiol. 2021;26: e12813.

Middlekauff HR, Vigna C, Verity MA, Fonarow GC, Horwich TB, Hamilton MA, Shieh P and Tupling AR. Abnormalities of calcium handling proteins in skeletal muscle mirror those of the heart in humans with heart failure: a shared mechanism? J Card Fail. 2012; 18:724-33.

Mielcarek A, Blauenburg B, Miethke M and Marahiel MA. Molecular insights into frataxin-mediated iron supply for heme biosynthesis in Bacillus subtilis. PLOS One. 2015; 10: e0122538.

Mishra S, Sabbah HN, Jain JC and Gupta RC. Reduced Ca2+-calmodulin-dependent protein kinase activity and expression in LV myocardium of dogs with heart failure. American journal of physiology Heart and circulatory physiology. 2003;284: H876-83.

Mitra A, Basak T, Datta K, Naskar S, Sengupta S and Sarkar S. Role of α-crystallin B as a regulatory switch in modulating cardiomyocyte apoptosis by mitochondria or endoplasmic reticulum during cardiac hypertrophy and myocardial infarction. Cell Death Dis. 2013;4: e582.

Myers L, Farmer JM, Wilson RB, Friedman L, Tsou A, Perlman SL, Subramony SH, Gomez CM, Ashizawa T, Wilmot GR, Mathews KD, Balcer LJ and Lynch DR. Antioxidant use in Friedreich ataxia. Journal of the neurological sciences. 2008;267:174-176.

Netticadan T, Temsah RM, Kawabata K and Dhalla NS. Sarcoplasmic reticulum Ca (2+)/Calmodulin-dependent protein kinase is altered in heart failure. Circ Res. 2000;86:596-605.

Norrish G, Rance T, Montanes E, Field E, Brown E, Bhole V, Stuart G, Uzun O, McLeod KA, Ilina M, Adwani S, Daubeney P, Delle Donne G, Linter K, Jones CB, Bharucha T, Cervi E and Kaski JP. Friedreich's ataxia-associated childhood hypertrophic cardiomyopathy: a national cohort study. Arch Dis Child. 2022; 107:450-455.

Panas M, Gialafos E, Spengos K, Papaioannou TG, Aggeli K, Kladi A, Siasos G, Gialafos J, Vassilopoulos D and Stefanadis C. Prevalence of interatrial block in patients with Friedreich's ataxia. Int J Cardiol. 2010; 145:386-387.

Pandolfo M. Molecular pathogenesis of Friedreich ataxia. Arch Neurol. 1999;56:1201-8.

Payne RM and Wagner GR. Cardiomyopathy in Friedreich ataxia: clinical findings and research. J Child Neurol. 2012;27:1179-86.

Payne RM. The Heart in Friedreich's Ataxia: Basic Findings and Clinical Implications. Prog Pediatr Cardiol. 2011;31:103-109.

Perdomini M, Belbellaa B, Monassier L, Reutenauer L, Messaddeq N, Cartier N, Crystal RG, Aubourg P and Puccio H. Prevention and reversal of severe mitochondrial cardiomyopathy by gene therapy in a mouse model of Friedreich's ataxia. Nature medicine. 2014;20:542-7.

Perry SW, Norman JP, Barbieri J, Brown EB and Gelbard HA. Mitochondrial membrane potential probes and the proton gradient: a practical usage guide. BioTechniques. 2011;50:98-115.

Peverill RE, Romanelli G, Donelan L, Hassam R, Corben LA and Delatycki MB. Left ventricular structural and functional changes in Friedreich ataxia-Relationship with body size, sex, age and genetic severity. PloS one. 2019; 14: e0225147.

Poulsen SH, Søgaard P, Nielsen-Kudsk JE and Egeblad H. Dilated Cardiomyopathy in Friedreich's Ataxia: 2D Echo and Tissue-Doppler Analysis of Left Ventricular and Atrial Function. European Journal of Echocardiography. 2003;4:331-333.

Pousset F, Legrand L, Monin M-L, Ewenczyk C, Charles P, Komajda M, Brice A, Pandolfo M, Isnard R, Tezenas du Montcel S and Durr A. A 22-Year Follow-up Study of Long-term Cardiac Outcome and Predictors of Survival in Friedreich Ataxia. JAMA Neurology. 2015;72:1334-1341.

Priori SG and Chen SRW. Inherited Dysfunction of Sarcoplasmic Reticulum Ca<sup>2+</sup> Handling and Arrhythmogenesis. Circulation research. 2011;108:871-883.

Puccio H, Simon D, Cossée M, Criqui-Filipe P, Tiziano F, Melki J, Hindelang C, Matyas R, Rustin P and Koenig M. Mouse models for Friedreich ataxia exhibit cardiomyopathy, sensory nerve defect and Fe—S enzyme deficiency followed by intramitochondrial iron deposits. Nat Genet. 2001;27:181-6.

Puccio H, Simon D, Cossée M, Criqui-Filipe P, Tiziano F, Melki J, Hindelang C, Matyas R, Rustin P and Koenig M. Mouse models for Friedreich ataxia exhibit cardiomyopathy, sensory nerve defect and Fe—S enzyme deficiency followed by intramitochondrial iron deposits. Nature Genetics. 2001;27:181-186.

Qaisar R, Bhaskaran S, Ranjit R, Sataranatarajan K, Premkumar P, Huseman K and Van Remmen H. Restoration of SERCA ATPase prevents oxidative stress-related muscle atrophy and weakness. Redox Biol. 2019;20:68-74.

Respress JL, Oort RJv, Li N, Rolim N, Dixit SS, deAlmeida A, Voigt N, Lawrence WS, Skapura DG, Skårdal K, Wisløff U, Wieland T, Ai X, Pogwizd SM, Dobrev D and Wehrens XHT. Role of RyR2 Phosphorylation at S2814 During Heart Failure Progression. Circulation research. 2012; 110:1474-1483.

Ristow M, Pfister MF, Yee AJ, Schubert M, Michael L, Zhang CY, Ueki K, Michael MD, 2nd, Lowell BB and Kahn CR. Frataxin activates mitochondrial energy conversion and oxidative phosphorylation. Proceedings of the National Academy of Sciences of the United States of America. 2000;97:12239-12243.

Roberge S, Roussel J, Andersson DC, Meli AC, Vidal B, Blandel F, Lanner JT, Le Guennec JY, Katz A, Westerblad H, Lacampagne A and Fauconnier J. TNF-α-mediated caspase-8 activation induces ROS production and TRPM2 activation in adult ventricular myocytes. Cardiovasc Res. 2014; 103:90-9.

Santos R, Lefevre S, Sliwa D, Seguin A, Camadro J-M and Lesuisse E. Friedreich ataxia: molecular mechanisms, redox considerations, and therapeutic opportunities. Antioxidants & redox signaling. 2010;13:651-690.

Santulli G, Xie W, Reiken SR and Marks AR. Mitochondrial calcium overload is a key determinant in heart failure. Proceedings of the National Academy of Sciences. 2015;112:11389-11394.

Scaduto RC, Jr. and Grotyohann LW. Measurement of mitochondrial membrane potential using fluorescent rhodamine derivatives. Biophysical journal. 1999;76:469-77.

Shannon TR, Pogwizd SM and Bers DM. Elevated Sarcoplasmic Reticulum Ca<sup>2+</sup> Leak in Intact Ventricular Myocytes From Rabbits in Heart Failure. Circulation research. 2003;93:592-594.

Shoshan-Barmatz V, De S and Meir A. The Mitochondrial Voltage-Dependent Anion Channel 1, Ca (2+) Transport, Apoptosis, and Their Regulation. Front Oncol. 2017;7:60.

Tsou AY, Friedman LS, Wilson RB and Lynch DR. Pharmacotherapy for Friedreich ataxia. CNS Drugs. 2009;23:213-23.

Tsou AY, Paulsen EK, Lagedrost SJ, Perlman SL, Mathews KD, Wilmot GR, Ravina B, Koeppen AH and Lynch DR. Mortality in Friedreich ataxia. J Neurol Sci. 2011;307:46-9.

Unverferth DV, Schmidt WR, Baker PB and Wooley CF. Morphologic and functional characteristics of the heart in Friedreich's Ataxia. The American journal of medicine. 1987;82:5-10.

Vangheluwe P, Schuermans M, Raeymaekers L and Wuytack F. Tight interplay between the Ca2+ affinity of the cardiac SERCA2 Ca2+ pump and the SERCA2 expression level. Cell Calcium. 2007;42:281-9.

Weidemann F, Liu D, Hu K, Florescu C, Niemann M, Herrmann S, Kramer B, Klebe S, Doppler K, Üçeyler N, Ritter CO, Ertl G and Störk S. The cardiomyopathy in Friedreich's ataxia-New biomarker for staging cardiac involvement. Int J Cardiol. 2015; 194:50-7.

Weidemann F, Rummey C, Bijnens B, Störk S, Jasaityte R, Dhooge J, Baltabaeva A, Sutherland G, Schulz JB and Meier T. The Heart in Friedreich Ataxia. Circulation. 2012;125:1626-1634.

Weisthal S, Keinan N, Ben-Hail D, Arif T and Shoshan-Barmatz V. Ca2+-mediated regulation of VDACI expression levels is associated with cell death induction. Biochimica et Biophysica Acta (BBA)-Molecular Cell Research. 2014;1843:2270-2281.

Yancey DM, Guichard JL, Ahmed MI, Zhou L, Murphy MP, Johnson MS, Benavides GA, Collawn J, Darley-Usmar V and Dell'Italia LJ. Cardiomyocyte mitochondrial oxidative stress and cytoskeletal breakdown in the heart with a primary volume overload. American journal of physiology Heart and circulatory physiology. 2015;308: H651-63.

Zorova LD, Popkov VA, Plotnikov EY, Silachev DN, Pevzner IB, Jankauskas SS, Babenko VA, Zorov SD, Balakireva AV, Juhaszova M, Sollott SJ and Zorov DB. Mitochondrial membrane potential. Anal Biochem. 2018;552:50-59.

What is claimed is:

1. A method of treating Friedrich's Ataxia in a subject in need thereof comprising administering to the subject a composition to upregulate type 2 ryanodine receptors (Ryr2), a composition to activate Ca²⁺ release, a composition to upregulate SERCA2a, a composition to upregulate Phasoplamban (PLN), a composition to upregulate Ca²⁺/calmodulin-dependent protein kinase II (CaMKII), or any combination thereof.

2. The method of claim 1, wherein the subject has a cardiac disease.

3. The method of claim 2, wherein the cardiac disease is hypertrophic cardiomyopathy, atrial tachycardia, intra atrial block, or any combination thereof.

4. The method of claim 1, wherein the subject exhibits reduced left ventricular ejection fraction.

5. The method of claim 1, wherein the composition to activate Ca²⁺ release comprises 4-chloro-m-cresol.

6. The method of claim 1, wherein the composition to upregulate SERCA2a comprises isatroxime, CDN1163, or any combination thereof.

7. The method of claim 1, wherein the composition to upregulate SERCA2a comprises a pyridine derivative.

8. The method of claim 1, wherein the composition to upregulate SERCA2a comprises AVVI/SERCA2a gene therapy.

9. The method of claim 1, wherein the composition to upregulate Ryr2 comprises mRNA gene therapy to overexpress Ryr2.

10. A method of diagnosing Friedrich's Ataxia in a subject comprising assaying Ryr2, SERCA2a, PLN, CaMKII, or any combination thereof from a sample from the subject;
and administering the composition of claim 1 to the subject wherein the subject has decreased Ryr2, PLN, CaMKII, and/or SERCA2a levels as compared to an unaffected control.

11. The method of claim 10, wherein assaying for Ryr2 comprises performing a Western blot test.

12. The method of claim 11, wherein performing the Western blot test comprises contacting the sample with an antibody, wherein the antibody is IgG kappa BP-HRP.

13. The method of claim 10, wherein the sample comprises a cell, a tissue, urine, blood, semen, plasma, feces, bone marrow, saliva, or any combination thereof from the subject.

14. The method of claim 10, wherein assaying the sample from the subject for Ryr2, PLN, CaMKII, and/or SERCA2a, or any combination thereof comprises analyzing the sample via mass spectrometry, performing an enzyme-linked immunosorbent assay (ELISA), performing a protein array, or performing a reverse-phase protein array.

15. A kit comprising (1) a device for screening a sample from a subject for Ryr2 and (2) a composition to upregulate Ryr2, a composition to activate Ca²⁺ release, a composition to upregulate SERCA2a, a composition to upregulate Phasoplamban (PLN), a composition to upregulate Ca²⁺/calmodulin-dependent protein kinase II (CaMKII) or any combination thereof.

16. The kit of claim 15, wherein the composition comprises 4-chloro-m-cresol, isatroxime, CDN1163, a pyridine derivative, AAVI/SERCA2a gene therapy, mRNA gene therapy to overexpress Ryr2, or any combination thereof.

17. The kit of claim 15, wherein the device for screening a sample comprises a Western blot test.

18. The kit of claim 15, wherein the subject has Friedrich's Ataxia.

19. The kit of claim 15, further comprising an antibody for a Western blot test, wherein the antibody is IgG kappa BP-HRP.

20. The kit of claim 15, wherein the sample comprises a cell or tissue from the subject.
